(19) United States
(12) Patent Application Publication
(10) Pub. No.: US 2008/0261824 A1
(43) Pub. Date: Oct. 23, 2008

(54) Rationale, methods, and assays for identifying novel taste cell genes and salty taste receptor targets and assays using these identified genes or gene products

(75) Inventors: Bryan Moyer, San Diego, CA (US); Min Lu, San Diego, CA (US); Fernando Echeverri, Chula Vista, CA (US); Dalia Kalabat, El Cajon, CA (US); Na Gao, San Diego, CA (US); Peter Hevezi, Encinitas, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Senomyx, Inc., San Diego, CA (US)

(21) Appl. No.: 11/808,356

(22) Filed: Jun. 8, 2007

Related U.S. Application Data
(60) Provisional application No. 60/811,763, filed on Jun. 8, 2006.
(60) Provisional application No. 60/831,199, filed on Jul. 17, 2006.
(60) Provisional application No. 60/848,995, filed on Oct. 4, 2006.
(60) Provisional application No. 60/866,178, filed on Nov. 16, 2006.

(51) Int. Cl. C40B 30/04; C12N 5/10; C12Q 1/68

(52) U.S. Cl. 506/9; 435/366; 435/6

(57) ABSTRACT

This invention relates to novel rationale and methods for identifying taste-specific genes, including genes involved in salty taste perception, especially human salty taste perception, but also genes involved in sweet, bitter, umami, and sour taste perception, and genes involved in other taste cell or taste receptor related activities such as digestive function and digestive related diseases, taste cell turnover, immunoregulation of the oral and digestive tract, and metabolic regulation such as in diabetes and obesity, the genes identified using these methods, and assays for identifying taste modulators (enhancers or blockers) and potential therapeutics using these genes. These compounds have potential application in modulating (enhancing or blocking) taste perception, especially salty taste perception and as potential therapeutics. In addition, this invention relates to novel methods for identifying taste-specific genes that can be used as markers for different taste cell types, including sweet, bitter, umami, sour, salt, and other taste cells in mammals as well as assays that measure the activity of the sweet, bitter, umami, or sour receptor in the presence of these genes to identify modulators of sweet, bitter, umami, and sour taste and to identify therapeutics especially for treating digestive or metabolic disorders, taste loss, and oral infections. Further, the invention provides specific methods of purifying, enriching, isolating or marking desired taste cell subtypes or lineages such as sweet, umami, bitter, salty, sour, fat or stem cells et al. e.g., by use of FACS, magnetic beads or other selection methods that purify, enrich, mark, or eliminate such as by use of labeled cytotoxins, cells that express or do not express one or more taste specific genes.

### RELATED APPLICATIONS

This application claims priority to U.S. provisional application Ser. No. 60/811,763 filed on Jun. 8, 2006; 60/831,199 filed on Jul. 17, 2006; 60/848,995 filed on Oct. 4, 2006; and 60/866,178 filed on Nov. 16, 2006. All of these provisional applications are incorporated by reference in their entirety herein.

### FIELD OF THE INVENTION

This invention in its broadest embodiment identifies a novel set of genes that are specifically expressed in chemosensory or more specifically taste cells, e.g., mouse circumvallate taste cells, and likely taste (e.g., circumvallate) cells derived from other mammals such as humans and non-human primates. These genes referred to herein as “taste-specific” genes because they are expressed specifically in taste cells. However, these taste-specific genes include genes which are directly or indirectly involved in taste detection and modulation, e.g., salty, umami, sweet, sour, fatty, metallic, or bitter taste transduction as well as including genes which are involved in biological functions not directly related to taste detection such as the modulation of digestion, taste cell turnover, regulation of the immune system, particularly of the oral cavity, and the regulation of metabolism e.g., carbohydrate metabolism, diabetes, obesity, cachexia, detection of food during digestion, et al.

Relating to the foregoing the present invention provides a novel set of genes that are expressed specifically in mouse chemosensory, e.g., mouse circumvallate taste cells that are not expressed or are expressed at significantly lower levels in lingual cells that are useful in screening assays, preferably high throughput screening assays, for identifying compounds that directly or indirectly modulate different taste modalities, e.g., salty, sweet, umami, bitter, sour, fatty, or metallic.

Further relating to the foregoing the present invention provides a novel set of genes and the corresponding gene products or cells that express same that are useful in screening assays, preferably high throughput screening assays, for identifying compounds that are useful e.g., as therapeutics in the treatment of digestive system disorders such as cancers and autoimmune disorders, for modulating taste cell apoptosis or taste cell turnover, for inducing taste cell regeneration, for affecting the regulation of immunity in the oral cavity, and the regulation of metabolism, e.g., in the treatment of diabetes, obesity, eating disorders, and other metabolic disorders.

Also relating to the foregoing the invention provides a novel set of genes which are useful in the identification and/or isolation and/or enrichment of specific types or lineages of taste or chemosensory cells, e.g., taste or chemosensory cells that are involved in specific taste modalities, immune system regulation in the oral cavity, taste cell apoptosis or taste cell turnover, taste cell regeneration, digestive system regulation, and the regulation of metabolism such as cells that aid in food detection, the secretion of hormones or enzymes involved in hunger and digestion, and the like.

Further, the invention relates to the use of these isolated chemosensory or taste cells in screening assays for identifying compounds that modulate taste, as well as in the identification of therapeutics for modulating the immune system, particularly the regulation of the immune homeostasis in the oral cavity, regulation of taste cell apoptosis, turnover or taste cell regeneration and proliferation, regulation of hormones or enzymes involved in digestion and other taste cell functions, treatment of digestive system disorders such as oral or digestive system cancers, autoimmune or inflammatory digestive disorders, treatment of diabetes, obesity, eating disorders, or other metabolic disorders, and the like.

More specifically, the present invention provides methods of isolating, purifying and marking desired taste cell types and taste cell lineages including e.g., umami, sweet, salty, bitter, fat, sour, metallic as well as taste stem cells and other taste cell lineages including cells that differentiate into taste bud cells, taste cell neurons, taste immune cells et al. based on the expression of one or more of the taste specific genes provided herein. These isolation and purification methods include both positive and negative cell separation methods. For example desired taste cell lineages or types may be isolated by positive cell selection methods e.g., by the use of fluorescence activated cell sorting (FACS), magnetic bead cell selection such as by visual identification of desired cells such as individual transfected cells by electrophysiology using antibody coated beads. Alternatively, desired taste cell lineages or types may be recovered or purified by negative cell purification and isolation methods wherein the desired cell types are enriched or purified from a mixed cell population by the removal of one or more undesired cell lineages e.g., by contacting a mixed cell population containing the desired taste cells and undesired cells with cytotoxic antibodies specific to a target gene or genes expressed on the undesired taste cell type(s) which are to be removed.

Also the invention relates to the use of markers e.g., antibodies or oligonucleotides specific to one or more of the subject taste specific genes in mapping regions of the tongue and oral cavity which are involved in specific taste and non-taste specific functions, mapping of specific regions of the gastrointestinal tract and associated organs that express specific taste specific genes and which therefore are involved in one or more of the taste cell specific functions disclosed herein, and/or the use of the subject genes in taste cell differentiation studies, .g. for identifying compounds that induce the differentiation of taste stem cells and other pluripotent or immature cell types into desired taste cell lineages and taste cell types.

This invention more specifically relates to novel rationale, methods, and assays including electrophysiological assays that identify and characterize novel taste-specific genes, including those that function as salt taste receptor targets. The invention also relates to the use thereof to identify modulators thereof, e.g., salty taste enhancers and the use thereof to modulate human salty taste perception and for treatment or prevention of conditions relating to sodium transport and absorption such as hypertension, hypotension, fluid retention, heart attack and stroke.

### BACKGROUND OF THE INVENTION

Epithelial sodium channels (ENaC) are members of the ENaC/degenerin family of ion channels that includes acid-sensing ion channels (ASIC) in mammals, mechanosensitive degenerin channels in worms, and FMRF-amide peptide-gated channels in mollusks (Kellenger, S. and Schild, L. (2002) Physiol. Rev. 82:735-767). ENaC mediates amiloride-sensitive apical membrane Na⁺ transport across high resistance epithelia in numerous tissues including kidney, colon, and lung.

ENaC is known to be a heterotrimeric channel comprised of alpha, beta, and gamma subunits or delta, beta, and gamma subunits. This heterotrimeric channel has been hypothesized to be involved in human salty taste perception. Previously, assays have been developed by the present assignee using ENaC sequences to identify compounds that modulate the delta beta gamma and alpha beta gamma human ENaC to examine if these compounds will potentially modulate human salty taste perception. Also, these compounds potentially may be used to treat human pathologies involving aberrant ENaC function.

Unlike other mammals, amiloride has been reported to only slightly reduce the intensity of sodium chloride taste, i.e., by about 15-20% when used at concentrations that specifically modulate ENaC function (Halpern, B. P. (1998) Neuroscience and Behavioral Reviews. 23: 5-47). Experiments conducted by the inventors have shown that amiloride, or the more potent amiloride derivative phenamil did not elicit a significant effect on perceived human salt intensity when tested at levels 300-fold (for amiloride) and 3000-fold (for benzamil) above IC50 values for alpha beta gamma ENaC (equivalent to 10-fold for amiloride and 100-fold for benzamil over IC50 values for delta beta gamma ENaC). Thus, additional non-ENaC genes are likely involved in human salt taste.

In addition, it has been recently reported that taste receptors may be expressed in non-oral tissues, e.g., in the digestive system and potentially other organs such as the kidney. Particularly it has been reported that sweet, bitter, and umami taste receptors are expressed in cells other than in the oral cavity such as gastrointestinal cells (See, e.g., Sternini et al., Amer J. Physiol. Gastrointestinal and Liver Physiology, 292:G457-G461, 2007; Mace, O. J. et al, J. Physiology. 10:1113/J. Physiol. 2007.130906. Published online May 10, 2007). Moreover, it is likely that salty receptors are expressed in the urinary tract. Based thereon, taste receptors are hypothesized to be involved in functions not directly related to taste such as digestive functions such as gastric motility, absorption, food detection, metabolism, and immune regulation of the oral or digestive tract and may also affect functions relating to sodium absorption, excretion and transport such as blood pressure and fluid retention. Therefore, the identification of taste cell specific genes and identifying what specific cells these genes are specifically expressed should facilitate a better understanding of other non-taste functions of these taste receptors and also facilitate the use of these genes, gene products and cells which express same in assays for identifying novel therapeutics, e.g., for treating digestive diseases such as autoimmune, inflammatory and cancers, metabolism, diabetes, eating disorders, obesity, taste cell turnover, hypertension, fluid retention, and immune regulation of the digestive system.

### BRIEF DESCRIPTION AND OBJECTS OF THE INVENTION

This invention in one embodiment relates to the identification of a novel set of genes that are expressed specifically in chemosensory or taste cells, particularly mouse circumvallate cells, and likely in taste cells of other mammals such as humans and non-human primates. These genes include genes which are directly or indirectly involved in detecting specific taste modalities such as salty, sweet, bitter, umami, sour, fatty and metallic taste and/or in modulating taste intensity and duration.

This invention in another embodiment relates to the identification of a novel set of genes that are expressed specifically in chemosensory or taste cells, particularly mouse circumvallate cells and likely in other chemosensory or taste cells and similar cells derived from other mammals such as humans and non-human primates that are involved in other taste cell functions including by way of example taste cell apoptosis or taste cell turnover, taste cell regeneration, digestion, regulation of the immune system in the oral cavity, regulation of carbohydrate or other metabolic functions relating to digestion, food detection, taste cell trafficking, and the like.

The invention in another embodiment further relates to the identification of specific genes or gene products expressed specifically in mouse or other mammalian taste cells that can be used as markers for the identification, isolation, or enrichment of specific taste cell subtypes or taste cell lineages including by way of example sweet, umami, sour, bitter, salty, fatty and metallic taste cells and for isolating taste cells that are involved in non-taste functions such as regulation of immunity, e.g., in the oral cavity, regulation of digestion or metabolism, regulation of taste cell apoptosis, turnover, or taste cell differentiation and proliferation, and regulation of sodium excretion, transport and absorption.

The invention in another embodiment further relates to the use of these taste cell specific genes or gene products or said isolated or enriched taste cell lineages or taste cell types expressing said taste cell specific genes for use in screening assays, e.g. for identifying compounds that elicit of modulate sweet, sour, umami, salty, bitter, fatty or metallic taste as well as the use of these genes, gene products, or isolated or enriched taste cells for the identification of potential therapeutic compounds, e.g., therapeutics for treatment of various digestive system disorders such as ulcerative colitis, Crohn's disease, celiac disease, dyspepsia, cancers of the digestive system, compounds for modulating taste cell turnover or apoptosis or for regulating taste cell differentiation and regeneration e.g., in geriatric subjects or individuals with cancer, or undergoing chemotherapy, or radiation, compounds for modulating or enhancing the immune system of the oral cavity, compounds for the regulation of digestion and metabolism, e.g., compounds that affect the production of digestive fluids, hormones or enzymes such as saliva, stomach and intestinal fluids, GLP-1 (glucagon-like peptide 1), GIP (glucose-dependent insulinotrophic polypeptide), secretin, amylase et al., compounds that affect digestive motility, compounds for treating diabetes, for modulating food detection, and compounds for treating obesity or eating disorders, cachexia, and the like.

The present invention further provides methods of isolating, purifying and marking desired taste cell types and taste cell lineages including e.g., umami, sweet, salty, bitter, fat, sour, metallic as well as taste stem cells and other immature and mature taste cell lineages including cells that differentiate into taste bud cells, taste cell neurons, taste immune cells et al. based on the expression or absence of expression of one or more of the taste specific genes provided herein. These isolation and purification methods include both positive and negative cell separation methods. For example desired taste cell lineages or types may be isolated by positive cell selection methods e.g., by the use of fluorescence activated cell sorting (FACS), magnetic bead cell selection e.g., by visual identification of desired cells such as individual transfected cells by electrophysiology using antibody coated beads. Alternatively, desired taste cell lineages or types may be recovered or purified by negative cell purification and isolation methods wherein the desired cell types are enriched or purified from a mixed cell population by the removal of one or several undesired cell lineages e.g., by contacting a mixed cell suspension containing the desired taste cells and undesired cells e.g., derived from the tongue, oral cavity or gastrointestinal tract and associated organs with cytotoxic antibodies specific to a target gene or genes expressed on the undesired taste cell type(s) which are to be removed.

Also the invention relates to the use of markers e.g., antibodies or oligonucleotides, that are specific to one or more of the subject taste specific genes provided herein in mapping regions of the tongue and oral cavity which are involved in specific taste and non-taste specific functions, mapping of cell comprised on specific regions of the gastrointestinal tract and associated organs such as the intestinal epithelium or urinary tract that express specific taste specific genes and which therefore are involved in one or more of the taste cell specific functions disclosed herein, and/or the use of the subject genes and markers specific thereto in taste cell differentiation studies, e.g. for identifying compounds that induce the differentiation or dedifferentiation of taste cells e.g., adult or embryonic stem cells and other pluripotent or immature cell types into desired taste cell lineages and taste cell types.

This invention in its more specific embodiments relates to novel rationale and methods, and results to date using these rationale and methods for identification and characterization of novel taste-specific genes that based on various parameters constitute salt receptor targets. The targets using these protocols are useful targets in high-throughput screening efforts to identify human salt taste enhancers. These targets were identified using two different techniques, gene chips and a polymerase chain reaction (PCR) screen, to identify novel salt receptor target genes. First, Affymetrix gene chips containing most all known mouse genes are used to determine which genes are specifically expressed in mouse circumvallate papilla taste cells at the back of the tongue and not lingual epithelial cells isolated by laser capture microdissection. Second, PCR is used to determine which ion channels, from 339 channels we have cataloged in the human/mouse genomes, are specifically expressed in human/mouse circumvallate (CV) papilla taste cells but not lingual epithelial cells isolated by laser capture microdissection. Taste-specific expression of genes identified by either approach, are confirmed using an independent histological method such as in situ hybridization or immunohistochemistry, to determine which genes are expressed in taste cells. Using double labeling histological methods, it is determined what novel taste-specific genes are expressed in sweet, bitter, and umami cells that express the taste-specific ion channel TRPM5, sour cells that express the taste-specific ion channel PKD2L1/PKD1L3, or a unique cell type that does not express TRPM5 or PKD2L1/PKD1L3. A taste-specific gene, preferably an ion channel, that is conductive or activated by sodium and is expressed in a TRPM5- and PKD2L1/PKD1L3-negative cell population is a probable candidate for screening efforts to identify the gene(s) that encode mammalian salty taste receptors, as well as specific cell types wherein these salty taste receptor genes are expressed such as in the oral cavity and urinary tract, and also for use in high throughput assays designed to identify enhancers of saltiness in humans.

While our efforts have been focused on identifying novel ion channels that may constitute salt receptor targets, we are cognizant of the fact that a salt receptor target may comprise another type of protein such as a transporter, a G-protein coupled receptor (GPCR), or an uncharacterized transmembrane protein. Therefore, we have also included membrane proteins with more than one transmembrane domain in our analyses. Since the sweet, bitter, umami, and sour receptors are all transmembrane proteins with multiple transmembrane domains, we rationalize that other taste receptors, including the salt receptor, would also be a protein with multiple transmembrane domains.

Also, though the focus of these experiments is to identify novel salt receptor targets, as discussed above it is reasonable to assume that during the course of our investigations that additional taste-specific genes involved in other taste modalities (for example sour taste, astringency, mouthfeel, fat taste, metallic taste, etc. . . . ) were identified. Thus, novel taste-specific genes identified herein as well as affecting salt perception (and other biological activities likely affected thereby such as sodium absorption, transport and excretion and the effects thereof such as fluid retention and blood pressure regulation) may alternatively affect other taste modalities and flavor perception in general. Additionally, though the focus of these experiments is to identify novel salt receptor targets, it is likely that during the course of our investigations that additional taste-specific genes will be identified. Thus, novel taste-specific genes identified herein could be used as specific markers of taste buds or taste receptor cell types including sweet, bitter, umami, sour, as well as salt cells and identified taste-specific genes could be targets for modulating the tastes of sweet, bitter, umami, sour, and salt.

Additionally, while the subject assays are designed to identify likely salt receptor targets it is further likely that these methods have identified taste-specific genes which are involved in non-taste biological functions such as discussed above. Therefore, these novel taste-specific genes and their corresponding gene products should be useful in isolating novel taste cell subtypes or taste cell lineages. In addition these taste-specific genes or the corresponding gene products or cells which express same such as the isolated or enriched endogenous taste or chemosensory cells which express these taste-cell specific genes are useful in therapeutic screening assays, e.g., for identifying therapeutics for the treatment of digestive system disorders such as digestive cancers, autoimmune and inflammatory digestive disorders such as ulcerative colitis, dyspepsia, Crohn's disease, celiac disease, inflammatory bowel syndrome, diverticulitis, et al., for regulating taste cell apoptosis or taste cell turnover, for inducing taste cell regeneration e.g. in geriatrics, cancer patients or individuals undergoing chemotherapy or radiation, for modulating the immune system of the oral cavity, for regulation of digestive mucous and fluids, enzymes or hormones such as GLP-1 (glucagon-like peptide 1), GIP (glucose-dependent insulinotrophic polypeptide), amylase, saliva, stomach acids, intestinal fluids, pepsin, secretin, and the like; for treatment of diabetes, eating disorders, cachexia, and other metabolic disorders involving these genes and/or isolated or enriched taste cells.

More particularly the present invention relates to the discovery of taste-specific genes that may play a role in taste cell development and apoptosis, taste cell regeneration, modulation of transcription factors that modulate taste cell receptor expression, taste receptor trafficking to and from the apical membrane/taste pore region, regulation of taste cell action potential firing frequency/membrane potential to control the intensity of and/or to modulate specific tastes, neurotransmitter release to afferent nerves that regulate taste intensity or specific tastes, and taste cell signaling to nerve fibers, et al.

Also the present invention further relates to the discovery of taste-specific genes specifically expressed in taste cells (which taste cells are e.g., in the digestive tract and the oral cavity, tongue, et al.), e.g., gastrointestinal cells or oral cavity cells, and the use of these genes, gene products or cells that express same to identify compounds that specifically bind to or which modulate the activity of these genes which compounds may be used to treat or prevent pathological conditions involving digestive function. These conditions include by way of example functional dyspepsia (bad digestion) and other dyspepsias which may or may not be ulcer derived or related and may involve different areas of the digestive tract such as the upper abdominal tract, the mid-abdominal tract or the lower abdominal tract.

Further the invention provides genes that are expressed specifically in taste cells such as salty or umami taste cells which genes, gene products or cells that express same e.g., gastrointestinal or oral cavity derived taste cells, may be used in screening assays to identify compounds that may be used to treat or prevent pathological conditions involving gastrointestinal fluids, mucous, enzymes or hormones involved with digestion or hunger such as gastrin, secretin, pepsin, cholecystokinin, glucagon-like peptide 1 (GLP-1), amylase, ghrelin, leptin and the like. Also these compounds may enhance the production of saliva or other digestive mucous secretions and fluids. These compounds potentially may be used to suppress or induce hunger and/or to modulate digestion in subjects in need thereof.

Further because the invention identifies genes specifically expressed in taste cells such as salty, sweet, bitter, sour or umami taste cells the invention also relates to the use of these genes, gene products, or cells that express same such as but not restricted to taste cells, e.g., gastrointestinal or oral cavity derived cells, in screening assays to identify compounds that bind to or modulate the activity or amount of these genes or gene products compounds which potentially may be used to treat or prevent pathological or chronic inflammatory or autoimmune gastrointestinal conditions such as Crohn's disease, inflammatory bowel syndrome (IBD), celiac disease, ulcerative colitis, diverticulitis, gastritis, reflux esophagitis, and the like. These compounds potentially may be used to treat or prevent autoimmune or inflammatory diseases affecting the digestive system.

Also, because the invention provides genes that are specifically expressed in taste cells, e.g., umami, sweet, salty, bitter, or sour taste cells, the invention further relates to the use of these genes, gene products or cells which express same such as taste cells, e.g., gastrointestinal or oral cavity derived taste cells in screening assays to identify compounds that bind to or modulate the activity of these genes or gene products which compounds potentially may be used to modulate gastric reflux and diseases or conditions associated therewith such as gastroesophageal reflux disease, heartburn, Barrett's esophagus, and esophagitis.

Also because the invention identifies genes that are specifically expressed in taste cells, e.g., umami, salty, sweet, bitter or sour cells the invention further relates to the use of these genes, gene products or cells which express same in screening assays to identify compounds that bind to or which modulate the activity of these genes or gene products and which therefore potentially may be used to treat or prevent cancers or malignancies associated with the digestive system such as by way of example cancers of the tongue, and oral cavity such as cancers of the taste buds and salivary gland cancers, stomach, esophagus, small or large intestine, anus or rectum, pancreas, gall bladder, liver, colorectal or colon.

Also because the invention identifies genes that are expressed specifically in taste cells, e.g., umami, sweet, sour or other taste cells the invention further relates to the use of these genes, gene products or cells which express same in screening assays to identify compounds that bind to or which modulate the activity of genes or gene products which compounds potentially my be use to treat or prevent appetite dysfunction and conditions associated therewith such as obesity, anorexia, bulimia, and cachexia associated therewith.

Also the invention relates to the use of the genes identified herein that are expressed specifically in taste cells for the isolation or enrichment of specific taste cell lineages or subtypes particularly taste cells derived e.g., from the tongue, oral cavity, or gastrointestinal system, which express one or several of these taste-cell specific genes.

Also because the invention identifies genes that are specifically expressed in taste cells (e.g. umami, sweet, sour, bitter or other taste cell types) and since taste cells are in the digestive tract and the oral cavity, tongue the invention further relates to the use of these genes, gene products and cells which express same such as but not restricted to taste cells, e.g., gastrointestinal cells or oral cavity cells, to identify compounds that bind to or which modulate the activity of these genes or gene products which may be used to treat or prevent pathological conditions involving digestive function. These conditions include by way of example functional dyspepsia (bad digestion) and other dyspepsias which may or may not be ulcer derived or related and may involve different areas of the digestive tract such as the upper abdominal tract, the mid-abdominal tract or the lower abdominal tract.

Further because the invention identifies genes that are specifically expressed in taste cells such as umami, sweet, sour, bitter, salty or other taste cells the invention further relates to the use of these genes, gene products and cells which express same such as but not restricted to taste cells, e.g., gastrointestinal or oral cavity cells, to identify compounds that may be used to treat or prevent pathological conditions involving gastrointestinal hormones, enzymes or fluids involved with digestion or hunger such as saliva, digestive fluids, gastrin, secretin, cholecystokinin, glucose-dependent insulinotrophic polypeptide, glucagon-like peptide 1, amylase, or ghrelin, leptin and the like. These compounds potentially may be used to suppress or induce hunger or to modulate digestion in subjects in need thereof.

Further because the invention provides genes that are specifically expressed in taste cells such as umami, sweet, sour, salty, bitter or other taste cells the invention also relates to the use of these genes, gene products, and cells which express same such as but not restricted to taste cells, e.g., gastrointestinal or oral cavity derived cells, in screening assays to identify compounds that bind to or modulate the activity of these genes or gene products which compounds potentially may be used to treat or prevent pathological or chronic inflammatory or autoimmune gastrointestinal conditions such as Crohn's disease, inflammatory bowel syndrome (IBD), celiac disease, ulcerative colitis, diverticulitis, gastritis, reflux esophagitis, and the like. These compounds potentially may be used to treat or prevent autoimmune or inflammatory diseases affecting the digestive system.

Also, because the invention identifies genes that are specifically expressed in taste cells, e.g., umami, sweet, salty, sour or other taste cells the invention further relates to the use of these genes, gene products or cells which express same such as taste cells, e.g., gastrointestinal or oral cavity derived cells in screening assays to identify compounds that bind to or modulate the activity of these genes which compounds that potentially may be used to modulate gastric reflux and diseases or conditions associated therewith such as gastroesophageal reflux disease, heartburn, Barrett's esophagus, and esophagitis.

Also because the invention provides genes that are specifically expressed in taste cells, e.g., umami, sweet, salty, bitter, sour or other taste cells the invention further relates to the use of these genes, gene products or cells which express same in screening assays to identify compounds that bind to or which modulate the activity of these genes and which compounds therefore potentially may be used to treat or prevent cancers or malignancies associated with the digestive system such as by way of example cancers of the salivary glands and taste buds, tongue, oral cavity, stomach, esophagus, small or large intestine, anus, pancreas, gall bladder, liver, colorectal, or colon.

Also because the invention identifies genes that are specifically expressed in taste cells, e.g., sweet, umami, bitter, sour, salty or other taste cells that are involved in sodium transport these genes, gene products, and the cells which express same can be used in screening assays for identifying compounds that regulate ion transport or ion flux, particularly sodium ions in order to identify therapeutic compounds that may be e.g., used to modulate blood pressure and fluid retention and conditions and diseases involving aberrant sodium absorption, excretion and transport.

Also because the invention identifies genes that are specifically expressed in taste cells, e.g., sweet, umami, bitter, sour, salty or other taste cells these genes, gene products, and the cells which express same can be used in screening assays for identifying compounds that regulate selective apoptosis of taste cells, modulation of transcription factors that control taste receptor expression, autocrine/paracrine modulation of taste cell development, taste bud lifetime, screens using genes that result in supertaster phenotypes, compounds that activate taste stem cells, compounds that affect trafficking of taste cell receptors e.g., from the apical membrane/taste pore region, compounds that affect taste intensity by modulating regulation of taste cell action via potential firing frequency/membrane potential, compounds that regulate neurotransmitter release to afferent nerves that control general or specific taste intensity, and autocrine/paracrine modulation of taste receptor function.

Also because the invention identifies genes that are specifically expressed in taste cells, e.g., sweet, umami, bitter, sour, salty or other taste cells these genes, gene products, and the cells which express same can be used in screening assays for identifying compounds that affect regeneration of taste cells or taste buds, e.g., in diseased or geriatric individuals or after injury or surgery, subjects undergoing chemotherapy or after injury, compounds for modulating drug-induced dysgeusia, ageusia, taste bud loss, dry mouth or xerostomia as for example found in Sjogren's syndrome, compounds that are useful in maintaining oral hygiene, treating or preventing halitosis, noxious oral microbia such as viruses and bacteria, and the like.

Also because the present invention identifies genes that are expressed specifically in taste cells, e.g., umami, sweet, bitter, salty, sour, fat, metallic et al., and other taste cell lineages such as stem cells, taste cell neurons, immune cells et al., the present invention also provides methods of isolating, purifying, enriching and marking desired taste cell types and taste cell lineages including e.g., umami, sweet, salty, bitter, fat, sour, metallic as well as taste stem cells and other taste cell lineages including cells that differentiate into taste bud cells, taste cell neurons, taste immune cells et al. based on the expression of one or more of the taste specific genes provided herein. These isolation and purification methods include both positive and negative cell separation methods. For example desired taste cell lineages or types may be isolated by positive cell selection methods e.g., by the use of fluorescence activated cell sorting (FACS), magnetic bead cell selection, e.g., by visual identification of desired cells such as individual transfected cells by electrophysiology using antibody coated beads. Alternatively, desired taste cell lineages or types may be recovered or purified by negative cell purification and isolation methods wherein the desired cell types are enriched or purified from a mixed cell population by the removal of undesired cell lineages e.g., by contacting a mixed cell population containing the desired taste cells and undesired cells with cytotoxic antibodies specific to a target gene or genes expressed on the undesired taste cell type(s) which are to be removed.

Also because the present invention identifies genes that are expressed specifically in taste cells, e.g., umami, sweet, bitter, salty, sour, fat, metallic, et al., and other taste cell lineages and types such as taste stem cells, taste neurons and taste immune cells the invention further relates to the use of markers e.g., antibodies or oligonucleotides, specific to one or more of the subject taste specific genes in mapping regions of the tongue and oral cavity which are involved in specific taste and non-taste specific functions, mapping of specific regions of the gastrointestinal tract and associated organs that express specific taste specific genes and which therefore are involved in one or more of the taste cell specific functions disclosed herein, and/or the use of the subject genes in taste cell differentiation studies, .g. for identifying compounds that induce the differentiation of taste stem cells and other pluripotent or immature cell types into desired taste cell lineages and taste cell types.

More specifically, as described in detail infra, the invention provides a rationale and criteria for a candidate salt taste gene, preferably an ion channel which exhibits:

a) Specific expression in taste cells and not lingual cells OR expression at higher levels in taste cells than lingual cells

b) Expression in a taste cell by histological methods. Specifically, expression in a unique taste cell type that does not express the sweet, bitter, and umami cell marker TRPM5 or the sour cell markers PKD2L1/PKD1L3. This unique cell type could be a dedicated salt sensing cell.

c) Functional expression as a sodium channel or a sodium-activated receptor with basal, constitutive function (i.e. a fraction of the channel population is open and passing sodium at rest) in heterologous expression systems (such as Xenopus oocytes and mammalian cells) or primary neurons (such as dorsal root ganglia neurons).

Genes fulfilling these criteria will be advanced into high-throughput screening efforts to identify compounds that enhance human salt perception. In addition the taste-specific genes reported herein, e.g., in Tables 1, 2 and 3 supra will be useful in the therapeutic screening assays as afore-mentioned.

Therefore in this patent application we describe screening assays to identify genes putatively involved in salty taste perception as well as taste and other taste-cell mediated activities in general.

It is a specific object to identify taste-specific genes encoding membrane proteins expressed specifically in taste cells and not lingual cells at higher levels in taste cells than lingual epithelial cells using gene chip and/or PCR methodologies and use same as salt receptor targets in assays to identify salty taste modulators as well as compounds that affect other taste modalities and taste perception and taste-cell related biological and cellular functions and taste cell related phenotypes in general.

It is a more specific object of the invention to determine which taste-specific genes are expressed in taste cells and especially in sweet, bitter, and/or umami cells (TRPM5 positive), sour cells (PKD2L1/PKD1L3 positive) or a unique cell type (TRPM5 negative). These unique cell types will likely comprise cells dedicated to salty taste perception.

It is another object of the invention to use these genes in specific assays to identify modulators (enhancers) of taste-specific ion channels or taste-specific genes as these compounds may modulate human salt taste perception.

It is a particular object of the invention to provide electrophysiological assays that measure conductance of putative taste ion channels identified herein in the presence and absence of putative enhancers.

It is another specific object of the invention to identify enhancers of the subject putative salty taste related ion channels and other taste affecting genes in an oocyte expression system.

It is a more specific object of the invention to provide patch clamping or two electrode voltage clamping assays using oocytes that express a putative salty taste receptor ion channel for identifying compounds that modulate the activity of this channel and therefore modulate salty taste. These and other objects of the present invention are met by one or more of the embodiments described below.

### SUMMARY OF THE INVENTION

This invention in its broadest embodiment identifies a set of genes that are specifically expressed in chemosensory, e.g., mouse circumvallate taste cells, and likely taste (e.g., circumvallate) cells derived from other mammals such as humans and non-human primates. These genes include genes which are directly or indirectly involved in taste detection and taste modulation, e.g., salty, umami, sweet, sour, fatty, metallic, or bitter taste transduction as well as functions not directly related to taste detection and taste modulation such as genes that are involved in the modulation of digestion and the production and composition of digestive fluids, mucous, enzymes and hormones such as saliva, stomach and intestinal fluids, GLP-1 (glucagon-like peptide 1), GIP (glucose-dependent insulinotrophic polypeptide), secretin, pepsin, and the like; genes that are involved in regulation of blood pressure and fluid retention, genes that are involved in taste receptor trafficking, taste cell turnover and taste cell regeneration, genes that are involved in the regulation of the immune system of the oral cavity and gastrointestinal system, genes that are involved in the prevention or onset of gastrointestinal related diseases such as cancers, inflammatory and autoimmune diseases affecting the oral cavity and digestive system, genes that are involved in the regulation of metabolism e.g., carbohydrate metabolism, obesity, eating disorders, genes that are involved in the detection of food during digestion, et al.

Relating to the foregoing the present invention provides a novel set of genes that are expressed specifically in mouse chemosensory, e.g., mouse circumvallate taste cells that are not expressed or are expressed at significantly lower levels in lingual cells that are useful in screening assays, preferably high throughput screening assays, for identifying compounds that directly or indirectly modulate different taste modalities, e.g., salty, sweet, umami, bitter, sour, fatty, or metallic.

Further relating to the foregoing the present invention provides a novel set of genes that are useful in screening assays, preferably high throughput screening assays for identifying compounds that are useful as therapeutics in the treatment of digestive system disorders, for modulating taste cell apoptosis or taste cell turnover, for inducing taste cell regeneration, for effecting the regulation of immunity in the oral cavity or digestive system, and the treatment of diabetes, obesity, eating disorders, and other metabolic disorders.

Also relating to the foregoing the invention provides a novel set of genes which are useful in the identification and/or isolation and/or enrichment of specific types or lineages of taste or chemosensory cells, e.g., taste or chemosensory cells that are involved in specific taste modalities, immune system regulation in the oral cavity, taste cell apoptosis or taste cell turnover, taste cell regeneration, digestive system regulation, and the regulation of metabolism such as by aiding in food detection, the secretion of hormones or enzymes involved in hunger and digestion, and the like.

Further, the invention relates to the use of the isolated chemosensory or taste cells in screening assays for identifying compounds that modulate taste, as well as in the identification of therapeutics for modulating the immune system regulation of the oral cavity, taste cell apoptosis turnover, taste cell regeneration, regulation of hormones or enzymes or fluids and mucous involved in digestion and other taste cell functions, treatment of digestive system disorders, treatment of diabetes, obesity, eating disorders, or other metabolic disorders, and the like.

Further, the present invention relates to methods of isolating, purifying and marking desired taste cell types and taste cell lineages including e.g., umami, sweet, salty, bitter, fat, sour, metallic as well as taste stem cells and other taste cell lineages including cells that differentiate into taste bud cells, taste cell neurons, taste immune cells et al. based on the expression of one or more of the taste specific genes provided herein. These isolation and purification methods include both positive and negative cell separation methods. For example desired taste cell lineages or types may be isolated by positive cell selection methods e.g., by the use of fluorescence activated cell sorting (FACS), magnetic bead cell selection, e.g., by visual identification of desired cells such as individual transfected cells by electrophysiology using antibody coated beads. Alternatively, desired taste cell lineages or types may be recovered or purified by negative cell purification and isolation methods wherein the desired cell types are enriched or purified from a mixed cell population by the removal of undesired cell lineages e.g., by contacting a mixed cell population containing the desired taste cells and undesired cells with cytotoxic antibodies specific to a target gene or genes expressed on the undesired taste cell type(s) which are to be removed.

Also, the invention relates to the use of markers or probes e.g., antibodies or oligonucleotides, which may be labeled with detectable markers such as radionuclides, fluorophores, enzymes and the like, which markers or probes are specific to one or more of the subject taste specific genes e.g., in mapping regions of the tongue and oral cavity comprising cells which are involved in specific taste modalities such as umami, sweet, bitter, salty, sour, fat, metallic, et al., as well as cells that are involved in non-taste specific functions such as are identified herein, mapping of specific regions of the gastrointestinal tract and associated organs that contain cells that express specific taste specific genes and which therefore are involved in one or more of the taste cell specific functions disclosed herein, and/or the use of the subject genes in taste cell differentiation studies, e.g. for identifying compounds that induce the differentiation or dedifferentiation of taste cells, e.g., adult or embryonic stem cells and other pluripotent or immature cell types into desired taste cell lineages and taste cell types.

This invention more specifically relates to novel rationale, methods, and assays including electrophysiological assays that identify and characterize novel taste-specific genes, including those that function as salty taste receptors.

It is believed that human salt taste may be mediated, in part, by a sodium or other ion channels as well as transporters and GPCRs expressed specifically in taste-cells. Thus, the invention provides methods for identifying taste-specific genes, including genes that may regulate salty taste, as well as other taste modalities and taste cell mediated functions and phenotypes using gene chip and PCR methodologies. The compounds identified and their derivatives that modulate the activity of these target genes potentially can be used as modulators of human salty taste in foods, beverages and medicinals for human consumption. Also, such compounds and their derivatives potentially may be used to treat diseases involving aberrant ion channel function. Further the compounds identified using the genes identified herein and cells which express same are useful in therapeutic screening assays as discussed herein for identifying potential therapeutics that modulate other taste-cell related functions and phenotypes.

In one mode, this invention provides a method for identifying a gene encoding a polypeptide in taste cells in a mammal. One embodiment of this method comprises the steps of (i) identifying a set of genes including genes which are expressed in taste cells but which are not expressed in lingual cells and/or genes which are expressed in taste cells at substantially higher levels than in lingual cells; (ii) identifying a subset of genes within the set of genes identified in (i) which are not expressed in taste cells which express umami, sweet or bitter taste receptors (T1Rs or T2Rs) or sour taste receptors (PKD2L1/PKD1L3); and (iii) functionally expressing one or more genes in the subset identified according to (ii) and determining which of these genes function as a sodium responsive ion channel or sodium responsive receptor or transporter and thereby identifying this gene or genes as a putative gene that modulates salty taste. Typically, the taste tissues for this method are derived from human or rodent source. In one preferred embodiment of the method, the genes in step (iii) function as sodium responsive ion channels, and more preferably, when the genes are expressed, a fraction of the channel population is open and passing sodium at rest.

In a preferred embodiment, step (i) comprises the use of laser capture microdissection (LCM) to dissect and purify taste tissues from non-taste tissues. In one mode of this embodiment, step (i) comprises RNA amplification of genes from taste cells and lingual cells and the amplified genes are screened against a gene chip containing a sample of genes specific to the particular mammal from which the taste and lingual tissues are obtained, and preferably, the gene chips include a set of annotated mammalian genes. In an alternative mode of this embodiment, step (i) comprises high throughput PCR using primers for each ion channel in a mammalian genome.

In another preferred embodiment, step (ii) is effected by in situ hybridization using antisense RNA probes specific for the set of genes identified in step (i) to determine level of expression in taste versus lingual cells. In an alternative preferred embodiment, step (ii) is effected by use of immunochemical detection using a labeled antibody specific to the protein encoded by gene or genes identified in step (i).

In another embodiment of the method for identifying a gene encoding a polypeptide involved in salty taste perception in a mammal, the method of this invention comprises the steps of (i) identifying a set of genes including genes which are expressed in taste cells but which are not expressed in lingual cells and/or genes which are expressed in taste cells at substantially higher levels than in lingual cells; (ii) identifying a subset of genes within the set of genes identified in (i) which are not expressed in taste cells which express umami, sweet or bitter taste receptors (T1Rs or T2Rs) or sour taste receptors (PKD2L1/PKD1L3); and (iii) determining, in a primary neuron which expresses one or more genes in the subset identified according to (ii), which of said genes functions as a sodium responsive ion channel or sodium responsive receptor or transporter and thereby identifying this gene or genes as a putative gene that modulates salty taste. In one mode of this embodiment, step (iii) comprises contacting the neuron with an antibody which specifically binds the gene and inhibits its function.

Genes identified according to either of the methods described above may be characteristic of cells which do not express TRPM5 and PKD2L1/PKD1L3. In another mode, this invention provides a method to assist in selecting cells which do not express TRPM5 and PKD2L1/PKD1L3 by determining whether a cell expresses a gene identified according to the methods above. Preferably, the gene used in the method of this paragraph is one of the genes listed in Tables 1-3, listing taste-specific genes encoding transmembrane proteins in taste cells. Efforts were focused on transmembrane genes since all known taste receptor genes for sweet, bitter, umami, and sour taste encode transmembrane proteins.

In another mode, this invention provides an assay for identifying a compound having potential in vivo application for modulating human salty taste. This method comprises the steps of (i) contacting a cell that expresses a gene encoding an ion channel, receptor or transporter identified as a putative salty taste affecting gene according to any one of the methods above, or a gene encoding a polypeptide possessing at least 90% sequence identity to the polypeptide encoded thereby, with at least one putative enhancer compound; (ii) assaying sodium conductance, receptor activity or sodium transport in the presence and absence of said putative enhancer; and (iii) identifying the compound as a potential salty taste enhancer based on whether it increases sodium conductance, the activity of said receptor or sodium transport. In various embodiments, the gene encodes an ion channel or the gene encodes a GPCR. Preferably, the gene is a human gene. More preferably, the method further includes testing the effect of the compound or a derivative thereof in a human taste test. Preferably, the selected compound promotes sodium ion transport into taste bud cells. The putative salty taste affecting gene may be expressed in an amphibian oocyte, or in a mammalian cell, preferably a Xenopus oocyte or a mammalian cell selected from the group consisting of a HEK293, HEK293T, Swiss3T3, CHO, BHK, NIH3T3, monkey L cell, African green monkey kidney cell, Ltk-cell and COS cell. Preferably, the putative salty taste affecting gene is expressed under the control of a regulatable promoter. The putative salty taste affecting gene may be expressed stably or transiently. In a preferred mode, the putative salty taste affecting gene is selected from the genes contained in tables 1-3 and their orthologs and variants.

In a preferred mode, the assay of step (ii) is an electrophysiological assay which uses a sodium sensitive dye, and preferred dyes include membrane potential dyes selected from the group consisting of Molecular Devices Membrane Potential Kit (Cat#R8034), Di-4-ANEPPS (pyridinium, 4-(2-(6-(dibutylamino)-2-naphthalen-yl)ethenyl)-1-(3-sulfopropyl)hydroxide, inner salt, DiSBACC4(2)(bis-(1,2-dibabituric acid)-triethine oxanol), Cc-2-DMPE (Pacific Blue 1,2-dietradecanoyl-sn-glycerol-3phosphoethanolamine, triethylammonium salt) and SBFI-AM (1,3-benzenedicrboxylic acid, 4,4-[1,4,10-trioxa-7,13-diazacylopentadecane-7,13-diylbis(5-methoxy-6,1,2-benzofurandiyl)}bis-tetrakis{(acetyloxy)methyl}ester (Molecular Probes), more preferably, the sodium sensitive dye is sodium green tetraacetate (Molecular Probes) or Na-sensitive Dye Kit (Molecular Devices). In another preferred mode, the assay of step (ii) is a two electrode voltage clamping assay in Xenopus oocytes, or the assay is a patch clamp assay in mammalian cells. Preferably, the assay measures activity by an ion flux assay, including using atomic absorption spectroscopy to detect ion flux.

Alternatively, the assay may use a fluorescence plate reader (FLIPR), or a voltage imaging plate reader (VIPR), which is used to increase ion channel-dependent sodium or fluid absorption. In a preferred embodiment of this method, the activity of the putative salty taste affecting gene is assayed in a frog oocyte electrophysiologically by patch clamping or two electrode voltage clamping, preferably using an automatic imaging instrument, which may be a fluorescence plate reader (FLIPR) or a voltage imaging plate reader (VIPR).

In yet another mode, this invention provides an assay for identifying a compound having potential in vivo application for modulating human sweet, bitter, umami, or sour taste. This method comprises the steps of (i) contacting a cell that expresses a gene in Tables 1-3 or an ortholog or variant with at least one putative enhancer or blocker compound; (ii) assaying sodium conductance, receptor activity or taste gene product function in the presence and absence of said putative enhancer or blocker; and (iii) identifying the compound as a potential enhancer or blocker for sweet, bitter or umami taste based on whether it modulates sodium conductance, the activity of said receptor or taste gene product function.

In yet another mode, this invention provides an assay for identifying a compound having potential in vivo application for as a potential therapeutic. This method comprises the steps of (i) contacting a cell that expresses a gene in Tables 1-3 or an ortholog or variant with at least one putative enhancer or blocker compound; (ii) assaying sodium conductance, receptor activity or taste gene product function in the presence and absence of said putative enhancer or blocker; and (iii) identifying the compound as a potential therapeutic that may be used to modulate a taste cell related function or phenotype that does not directly involve taste such a digestive disorder or disease, taste cell or taste bud turnover or regeneration, immune regulation of the oral or digestive system, or treatment of a metabolic disorder such as diabetes, obesity, eating disorder et al., based on whether it modulates sodium conductance, the activity of said receptor or taste gene product function.

### BRIEF DESCRIPTION OF THE FIGURES

FIG. 1 contains an example of LCM using human taste tissues.

FIG. 2 contains an example of PCR quality control of human taste and lingual cells.

FIG. 3 contains an example of high-throughput PCR screening to identify novel taste-specific ion channels.

FIG. 4 contains an example of in situ hybridization and immunochemistry histological methods to visualize expression of the known taste gene gustducin in mouse or human taste tissue sections.

FIG. 5 contains an example of in situ hybridization and immunochemistry histological methods to visualize TRPM5 taste gene expression in mouse or human taste tissue sections.

FIG. 6: Example of immunohistochemistry to visualize TRPM5 taste gene expression in mouse taste tissue.

FIG. 7: Example of immunohistochemistry histological method to visualize SCN3A/Nav1.3 sodium channel gene expression in mouse taste tissue.

FIG. 8: Example of double labeling immunohistochemistry illustrating coexpression of SCN3A and TRPM5 in the same taste cells.

FIG. 9: Example of immunohistochemistry to visualize PKD2L1 taste gene expression in mouse taste tissue.

FIG. 10: Example of double labeling immunohistochemistry illustrating PKD2L1 and TRPM5 expression in different taste cells.

FIG. 11 contains a double labeling experiment that visualizes HCN4 and TRPM5 expression in separate mouse CV cells.

FIG. 12 contains a double labeling immunochemistry experiment visualizing HCN4 and PKD2L1 expression in the same mouse CV taste cells.

FIG. 13 contains a double labeling experiment demonstrating HCN4 exclusion from the taste pore in mouse CV taste cells.

### DETAILS OF THE INVENTION

The invention relates to the identification of genes expressed specifically in taste tissues which are putatively involved in salty taste or other taste modalities or taste in general; or which are involved in taste cell related functions and phenotypes that do not directly involve taste such as taste cell or taste bud regeneration and turnover, immunoregulation of the oral cavity or digestive system, regulation of digestion or metabolism, onset or prevention of digestive system disorders such a cancers, autoimmune diseases, and inflammatory conditions such as IBD, ulcerative colitis, Sjogren's syndrome, celiac disease, Crohn's disease, and the like and the use thereof in screening assays to identify compounds that modulate salty taste perception or other taste modalities or taste in general or for identifying potential therapeutics for use in humans. In particular the invention includes use of the following methodologies, to identify novel taste-specific genes:

1) Laser capture microdissection (LCM) and RNA amplification. In laser capture microdissection, a fine laser beam is used to dissect and purify taste cells from histological sections. This method isolates taste cells, devoid of contaminating lingual epithelial cells and connective tissue, and allows one to perform molecular biology experiments on a highly enriched taste cell population. In parallel, lingual epithelial cells are isolated by LCM and used as a negative control devoid of taste cells. LCM is advantageous to manual or enzymatic dissection of taste papilla because these crude techniques yield a heterogeneous mixture of taste and lingual cells in which taste cells comprise 1-20% of collected material. RNA amplification amplifies total RNAs from taste cells and lingual cells isolated by LCM up to 1 million-fold in a non-biased fashion to generate sufficient genetic material to perform molecular biology studies (gene chips or PCR). We have found that 1300-2000 taste cells are sufficient for gene chip experiments with mouse taste tissue and greater than 5,000 taste cells are sufficient for PCR experiments with human taste tissue.

2) Gene chips—Gene chips contain most all annotated genes on a small chip. Hybridizing RNA, isolated and amplified from taste and lingual cells, to gene chips can be used to determine which specific genes are expressed in taste cells and not lingual cells and which specific genes are expressed at higher levels in taste cells compared to lingual cells.

3) PCR—high-throughput PCR is performed in 96 well plates using primers specific for each ion channel in the human/mouse genome and amplified RNA from human/mouse taste and lingual cells isolated by LCM. Detection of products of the appropriate size in taste cells but not lingual cells and DNA sequencing of the products to confirm gene identity indicates the ion channel of interest is a taste-specific gene.

4) In situ hybridization—antisense RNA probes specific for an individual gene(s) (identified by gene chips or PCR) are hybridized to tissue sections containing taste cells to determine if the mRNA transcript for the gene of interest is expressed in taste cells, specifically in sour, sweet, bitter, and/or umami cells or in a unique cell type that may be involved in salt taste detection.

5) Immunohistochemistry—antibodies specific for an individual protein (whose gene was identified by gene chips or PCR) are applied to tissue sections containing taste cells to determine if the protein of interest is expressed in taste cells, specifically in sour, sweet, bitter, and/or umami cells or in a unique cell type that may be involved in salt taste detection.

RNA Quality Control

RNA integrity is gauged using two methods. RNA quality is determined, using an Agilent 2100 Bioanalyzer (Agilent Technologies) with a Series II RNA 6000 Pico Assay (Cat # 5067-1514). The ratio between the abundant 28S ribosomal RNA (rRNA) and 18S rRNA bands is quantitated. A ratio of 2 indicates high quality RNA with no degradation. A ratio ˜1 indicates partial RNA degradation. Ratios between 0.5-1.0 are commonplace for LCM samples due to partial RNA degradation that occurs when samples are stained and processed for LCM at room temperature. In addition, the RNA Integrity Number (RIN) is determined. A RIN value of 10 indicates high quality RNA with no degradation, a RIN value of 5 indicates partial RNA degradation and a RIN of 1 indicates massive RNA degradation. RIN values >5 are acceptable for gene chip analysis and are commonplace for LCM samples. Typically 28S/18S rRNA ratios were ˜1.0 and RIN values were 7-8 for mouse taste tissue samples whereas 28S/18S rRNA ratios were 0.5-1.0 and RIN values were 5-6 for human taste tissue samples.

Second, RNA quantity is determined with the Quant-iT RiboGreen RNA Assay Kit (Molecular Probes, Cat # R11490). A fluorescent RNA binding dye, with sensitivity down to 1 pg/ul, is used to quantitate the amount of total RNA in taste and lingual cell samples. Precise quantitation of RNA is important for adding equal amounts of taste and lingual RNA into gene chip and PCR experiments.

2) Gene Chips:

Gene chips experiments were conducted on 5 paired mouse CV taste and lingual samples using Affymetrix 430 2.0 arrays and analyzed using GeneSpring GX v7.3 software (Agilent Technologies). Between 1300-2000 CV taste and lingual cells were separately isolated by LCM and total RNA was purified for each sample. RNA was then amplified and hybridized to gene chips. Data analyses were performed using two separate algorithms: Affymetrix Microarray Suite 5 (MAS5) which takes into account both perfect match and mismatch probes on gene chips, and robust multi-chip algorithm (RMA) which only takes into account perfect match probes on gene chips. Taste-specific genes encoding transmembrane proteins were identified in this analysis.

3) PCR: Prior to high-throughput PCR using primers against all 339 ion channels identified in the human/mouse genomes, quality-control PCR reactions are first performed on up to 4 known taste-specific genes and 2 housekeeping genes to ensure that taste and lingual RNAs are of high quality. The 4 taste-specific genes examined are the G alpha protein gustducin, the sweet receptor component T1R2, the ion channel TRPM5, and the enzyme phospholipase C isoform beta2; the two housekeeping genes examined are beta-actin and GAPDH. Specific expression of the taste genes by taste cells but not lingual cells plus expression of the ubiquitous housekeeping genes by both taste and lingual cells indicates high quality RNA material.

PCR products are analyzed on agarose gels to determine if bands of the appropriate size are present in taste cells but not lingual cells. Genes with this expression pattern are putative taste-specific genes. All taste-specific genes were cloned and sequenced to confirm the gene identities.

4) In Situ Hybridization:

In double labeling in situ hybridization, two different RNA probes are generated to label two different genes, specifically two different taste-specific genes identified by gene chip and/or PCR approaches. Alternatively, one probe can be generated to label a single gene to determine if the gene is expressed in taste cells. For double labeling studies, the first gene is labeled with a FITC probe that generates one color in a fluorescent microscope while the second gene is labeled with a digoxygenin (DIG) probe that generates a different color in a fluorescent microscope. Superimposition of probe 1 and probe 2 reveals if genes are expressed in the same or in different cell types. For example, if a unique ion channel identified by gene chip or PCR approaches colocalizes to cells expressing TRPM5, that unique ion channel is expressed in cells responsible for sweet, bitter, and/or umami taste. By contrast, if a unique ion channel identified by gene chip or PCR approaches does not colocalize to cells expressing TRPM5, that unique ion channel is expressed in a different cell type that may be responsible for salt taste (or another taste modality) and that unique ion channel may be directly involved in sodium detection.

5) Immunohistochemistry:

In double labeling immunohistochemistry, two different antibody probes are used to label two different proteins, specifically two different taste-specific proteins whose genes were identified by gene chip and/or PCR approaches. Alternatively, one antibody probe can be used to label a single protein to determine if the protein is expressed in taste cells. For double labeling studies, the first protein is labeled with an antibody at a very dilute concentration that can only be detected with a sensitive detection method termed tyramide signal amplification (TSA). The second protein is then labeled with another antibody and detected using a non-TSA method. The dilute first antibody cannot be detected by the standard non-TSA method; therefore, two different antibodies from the same species (e.g. rabbit polyclonal antibodies) will not cross-react and, therefore, can be used in double labeling experiments. Superimposition of protein label 1 and protein label 2 reveals if proteins are expressed in the same or in different cell types. For example, if a unique ion channel identified by gene chip or PCR approaches colocalizes to cells expressing TRPM5, that unique ion channel is expressed in cells responsible for sweet, bitter, and/or umami taste. By contrast, if a unique ion channel identified by gene chip or PCR approaches does not colocalize to cells expressing TRPM5, that unique ion channel is expressed in a different cell type that may be responsible for salt taste (or another taste modality) and that unique ion channel may be directly involved in sodium detection.

Using such rationale, methods and protocols the following genes were identified which are contained in the Tables herein. These Tables are briefly described as follows.

Table 1: Summary of mouse taste-specific genes encoding transmembrane proteins from Affymetrix 430 2.0 microarrays/gene chips.

Table 2: Summary of human and mouse taste-specific ion channels from PCR screens.

Table 3: Summary of colocalization of taste-specific genes in TRPM5 (sweet, bitter, umami) and PKD2L1/PKD1L3 (sour) cells.

Therefore, based on the foregoing, the subject invention generally relates to methods for identifying taste genes, including genes involved in salty taste perception and the use in screening assays for identifying human salty taste enhancers and other taste modulatory compounds and for identifying potential therapeutics that modulate other taste cell related functions and phenotypes including diseases and conditions not directly related to taste transduction.

Particularly, the present invention includes the use of cell-based assays to identify salty taste modulators (enhancers). These compounds have potential application in modulating human salty taste perception. Compounds identified for example in electrophysiological assays and their biologically acceptable derivatives are to be tested in human taste tests using human volunteers to confirm their effect on human salty taste perception. In addition compounds identified as potential therapeutics will be evaluated in appropriate in vitro and in vivo models depending on the nature of the intended application. For example compounds identified as potential therapeutics for diabetes may be evaluated in well known diabetic animal models such the NOD mouse model or BB rat model. Similarly, compounds identified as potential therapeutics for IBD or Crohn's disease may be tested in rodent animal models for IBD or Crohn's disease.

As discussed further infra, the cell-based assays used to identify taste, e.g., salty taste modulatory or therapeutic compounds will preferably comprise high throughput screening platforms to identify compounds that modulate (enhance) the activity of genes involved in salty taste perception using cells that express the genes disclosed herein or combinations thereof. Additionally, these sequences may be modified to introduce silent mutations or mutations having a functional effect such as defined mutations that affect ion (sodium) influx. As noted above, the assays will preferably comprise electrophysiological assays effected in amphibian oocytes or assays using mammalian cells that express a an ion channel according to the invention using fluorescent ion sensitive dyes or membrane potential dyes, e.g., sodium-sensitive dyes. Preferably, compounds that modulate such ion channels are identified by screening using electrophysiological assays effected with oocytes that express an ion channel identified herein (e.g., patch clamping or two electrode voltage clamping).

Still alternatively, compounds that modulate the subject ion channels putatively involved in salty taste may be detected by ion flux assays, e.g., radiolabeled-ion flux assays or atomic absorption spectroscopic coupled ion flux assays. As disclosed supra, these compounds have potential application in modulating human salty taste perception or for modulating other biological processes involving aberrant or normal ion channel function.

The subject cell-based assays use mutant nucleic acid sequences which are expressed in desired cells, preferably oocytes or human cells such as HEK-293 cells, or other human or mammalian cells conventionally used in screens for identifying ion channel or GPCR modulatory compounds. These cells may further be engineered to express other sequences, e.g., other taste GPCRs, i.e., T1Rs or T2Rs such as are described in other patent applications by the present Assignee Senomyx as well as appropriate G proteins. The oocyte system is advantageous as it allows for direct injection of multiple mRNA species, provides for high protein expression and can accommodate the deleterious effects inherent in the overexpression of ion channels. The drawbacks are however that electrophysiological screening using amphibian oocytes is not as amenable to high throughput screening of large numbers of compounds and is not a mammalian system. As noted, the present invention embraces assays using mammalian cells, preferably high throughput assays.

Some ion channels putatively involved in salty taste (ENaC) proteins are known to form heteromeric channels comprised of three subunits, an alpha, beta, and a gamma or delta subunit. The sequences of these respective ENaC subunits are disclosed in an earlier patent application by the present Assignee, U.S. Ser. No. 10/133,573 which is incorporated by reference in its entirety herein. Upon co-expression in a suitable cell these subunits result in a heterotrimeric channel having cation ion channel activity; in particular it responds to sodium and should similarly respond to lithium ions in cell-based assays such as those which are disclosed herein and in Senomyx's prior application referenced above.

The Senomyx applications incorporated by reference provides high throughput screening assays using mammalian cells transfected or seeded into wells or culture plates wherein functional expression in the presence of test compounds is allowed to proceed and activity is detected using membrane-potential fluorescent or ion (sodium) fluorescent dyes.

As discussed above, the invention specifically provides methods of screening for modulators, e.g., activators, inhibitors, stimulators, enhancers, etc., of human salty taste or other taste modalities and potential therapeutics that target other taste cell functions or phenotypes using the nucleic acids and proteins, sequences provided herein. Such modulators can affect salty taste or other taste modalities or taste cell related functions and phenotypes, e.g., by modulating transcription, translation, mRNA or protein stability; by altering the interaction of the ion channel with the plasma membrane, or other molecules; or by affecting ion channel protein activity. Compounds are screened, e.g., using high throughput screening (HTS), to identify those compounds that can bind to and/or modulate the activity of a taste receptor or taste ion channel polypeptide or transporter or fragment thereof. In the present invention, proteins are recombinantly expressed in cells, e.g., human cells, or frog oocytes and the modulation of activity is assayed by using any measure of ion channel, receptor or transporter function, such as measurement of the membrane potential, or measures of changes in intracellular sodium or lithium levels. Methods of assaying ion, e.g., cation, channel function include, for example, patch clamp techniques, two electrode voltage clamping, measurement of whole cell currents, and fluorescent imaging techniques that use ion-sensitive fluorescent dyes and ion flux assays, e.g., radiolabeled-ion flux assays or ion flux assays.

Further, as afore-mentioned, the present invention further provides methods of isolating, purifying and marking desired taste cell types and taste cell lineages including e.g., umami, sweet, salty, bitter, fat, sour, metallic as well as taste stem cells and other taste cell lineages including cells that differentiate into taste bud cells, taste cell neurons, taste immune cells et al. based on the expression of one or more of the taste specific genes provided herein. These isolation and purification methods include both positive and negative cell separation methods. For example desired taste cell lineages or types may be isolated by positive cell selection methods e.g., by the use of fluorescence activated cell sorting (FACS), magnetic bead cell selection, e.g., by visual identification of desired cells such as individual transfected cells by electrophysiology using antibody coated beads. Alternatively, desired taste cell lineages or types may be recovered or purified by negative cell purification and isolation methods wherein the desired cell types are enriched or purified from a mixed cell population by the removal of undesired cell lineages e.g., by contacting a mixed cell population containing the desired taste cells and undesired cells with cytotoxic antibodies specific to a target gene or genes expressed on the undesired taste cell type(s) which are to be removed.

For example, the taste specific genes identified herein reported in Tables 1, 2 and 3 can be used as markers to identify and/or purify specific taste cells including by way of example sweet, umami, sour, bitter, salty, fat and other taste cells including stem cells. In one embodiment, antibodies directed against at least one of the proteins encoded by a gene contained in one of Tables 1, 2 or 3, or an ortholog or variant thereof, e.g., a variant encoding a protein at least 90% identical thereto, can be used to label cells contained in a suspension of taste cells, e.g., taste bud cells, or a suspension of gastrointestinally derived cells including taste cells e.g., produced by enzymatic digestion and tissue disaggregation (See e.g., Herness M. A., Neuroscience Letters 106:60-64 (1989) et al. which teaches a disassociation procedure for isolating mammalian taste buds). Also, the separation of desired taste cell lineages or subtypes can be achieved by the use of a fluorescence cell activated sorter (FACS) (See e.g., Beavis, A J and Pennline K J Biotechniques 21:498-503 (1996)), or by use of a magnetic bead cell separation techniques (see e.g. Jurman et al., Biotechniques 17:887-881 (1994) which reference reports the visual identification of transfected cells using antibody coated beads) or by the use of other methods generally known in the art for isolating, purifying, marking and/or enriching desired cells contained in a mixed cell population based on the expression of one or several marker genes. Also, cells belonging to a specific cell subset can be purified or enriched by negative cell selection procedures which eliminate non-target cells e.g., those representing non-target taste cell subsets, by the use of methods that eliminate non-target cells, e.g., by contacting the mixed cell population with cytotoxic antibodies specific to one or several non-target taste cell subtypes.

Also, the invention provides methods of using the subject taste specific genes contained in Table 1, 2, or 3, or orthogs and variants thereof and the use of markers or probes specific thereto, e.g., antibodies or oligonucleotides, which may be labeled with detectable markers such as radionuclides, fluorophores, enzymes and the like, which markers or probes are specific to one or more of the subject taste specific genes, for mapping specific regions of the tongue and oral cavity that comprise cells which are involved in specific taste modalities such as umami, sweet, bitter, salty, sour, fat, metallic, et al., as well as for mapping cells that are involved in non-taste specific functions such as are identified herein, and for mapping of specific regions of the gastrointestinal tract and associated organs that contain cells that express specific taste specific genes and which therefore are involved in one or more of the taste cell specific functions disclosed herein, and/or the use of the subject genes in taste cell differentiation studies, e.g. for identifying compounds that induce the differentiation or dedifferentiation of taste cells, e.g., adult or embryonic stem cells and other pluripotent or immature cell types into desired taste cell lineages and taste cell types.

This invention more specifically relates to novel rationale, methods, and assays including electrophysiological assays that identify and characterize novel taste-specific genes, including those that function as salty taste receptors.

It is believed that human salt taste may be mediated, in part, by a sodium or other ion channels as well as transporters and GPCRs expressed specifically in taste-cells. Thus, the invention provides methods for identifying taste-specific genes, including genes that may regulate salty taste, as well as other taste modalities and taste cell mediated functions and phenotypes using gene chip and PCR methodologies. The compounds identified herein and their derivatives which bind to and/or modulate the function of the taste specific genes provided herein are useful as taste modulators and also as therapeutics e.g., for treating gastrointestinal and metabolic disorders such as diabetes, obesity, cachexia and the like.

### Definitions

“Putative salty taste receptor or ion channel gene” refers to a gene specifically expressed in taste cells that is not expressed in lingual cells or is expressed substantially less in lingual cells that moreover preferably is not expressed in taste cells that express a T1R, T2R, TRPM5, or PKD2L1/PKD1L3 gene.

“Taste Cell” refers to a cell that when mature expresses at least one receptor, transporter, or ion channel that directly or indirectly regulates or modulates a specific taste modality such as sweet, sour, umami, salty, bitter, fatty, metallic or other taste perception or general taste perception such as taste intensity or the duration of a taste response. Taste cells express mRNA and/or a protein for the gene C6orf15 (chromosome reading frame 15)-also known as STG. This gene has been described as a taste-specific gene (M. Neira et al., A New Gene (mSTG) specific for taste buds is found by laser capture microdissection. Mammalian Genome 12:60-66 (2001)) and is among the mouse taste specific genes reported herein. In addition a mature taste receptor cell typically will express mRNA and/or protein for alpha ENaC. We have data (not shown herein) that reveals that alpha ENaC is expressed in at least sweet, bitter, umami, sour and most likely salty taste cells. Further, a mature taste receptor cell will typically express mRNA and/or protein for cytokeratin 19. This protein is only expressed in mature taste cells and is not found in basal or stem cells. (L. Wong et al., “Keratin-like immunoreactivity in receptor cells of mammalian taste buds”. Chemical Senses 19(3):251-264 (1994)). Furthermore, taste cells can be identified by those skilled in the art based on their characteristic morphology. In particular mature taste receptor taste cells are elongated and spindle-shaped. Also, a mature taste receptor cell has the apex of the cell (apical membrane) penetrating into the taste pore thereby gaining access or exposure to saliva. By contrast, an immature taste cell, e.g., a basal cell or stem cell is rounded and is not exposed to the taste pore and saliva. Also, unlike mature taste cells, basal and stem cells tend to be localized towards the base of taste buds.

“Chemosensory cells” are cells that are involved in sensing of chemical stimulants such as tastants and other chemical sensory stimuli such as odorants. Chemosensory cells herein include in particular taste receptor cells and cells comprised in the digestive or urinary tract or other organs that when mature express one or more taste receptors. For example, gastrointestinal chemosensory cells are known which express T1Rs or T2Rs and which cells are likely involved in food sensing, metabolism, digestion, diabetes, food absorption, gastric motility, et al. In addition, cells found in the urinary tract likely express salty taste receptors and are involved in sodium transport, excretion and functions associated therewith such as blood pressure and fluid retention. Further, in the digestive system chemosensory cells that express taste receptors may also express chromogranin A, which is a marker of secretory granules. (C. Sternini, “Taste Receptors in the Gastrointestinal Tract. IV. Functional Implications of Bitter Taste Receptors in Gastrointestinal Chemosensing”. American Journal of Physiology, Gastrointestinal and Liver Physiology.”, 292:G457-G461, 2007).

“Taste-cell specific gene” herein refers to a gene specifically expressed by a taste cell, e.g., a circumvallate taste cell that is not specifically expressed by lingual cell that is involved in a taste or non-taste related taste cell function or phenotype. Taste cells include cells in the oral cavity that express taste receptors such as the tongue and taste cells in other areas of the body that express taste receptors such as the digestive system and urinary tract. Such genes include those comprising the Accession Numbers contained in Tables 1, 2 and 3, as well as orthologs thereof, allelic variants, chimeras, and genes that hybridize thereto under stringent hybridization conditions and/or genes which encode a protein possessing at least 80%, more preferably at least 90%, and even more preferably at least 95% to any of the foregoing.

These taste-cell specific genes include genes involved in taste and non-taste related functions such a taste cell turnover, diseases affecting the digestive system or oral cavity, immunoregulation of the oral cavity and/or digestive system, digestive and metabolic functions involving taste cells such a diabetes, obesity, blood pressure, fluid retention et al. In referring to the particular taste specific genes identified herein as noted these genes include the nucleic acid sequences corresponding the Accession Numbers contained in Tables 1, 2, and 3 as well as orthologs thereof and chimeras and variants including allelic variants thereof. In particular such variants include sequences encoding polypeptides that are at least 80% identical, more preferably at least 90% or 95% identical to the polypeptides encoded by the genes corresponding to the recited Accession numbers or to orthologs thereof, especially human and non-human primate orthologs. In addition, the genes include nucleic acid sequences that hybridize under stringent hybridization conditions to a nucleic acid sequence corresponding to one of the gene sequences corresponding to the gene Accession numbers recited in the Tables herein.

“Cation channels” are a diverse group of proteins that regulate the flow of cations across cellular membranes. The ability of a specific cation channel to transport particular cations typically varies with the valency of the cations, as well as the specificity of the given channel for a particular cation.

“Homomeric channel” refers to a cation channel composed of identical alpha subunits, whereas “heteromeric channel” refers to a cation channel composed of two or more different types of alpha subunits. Both homomeric and heteromeric channels can include auxiliary beta subunits.

A “beta subunit” is a polypeptide monomer that is an auxiliary subunit of a cation channel composed of alpha subunits; however, beta subunits alone cannot form a channel (see, e.g., U.S. Pat. No. 5,776,734). Beta subunits are known, for example, to increase the number of channels by helping the alpha subunits reach the cell surface, change activation kinetics, and change the sensitivity of natural ligands binding to the channels. Beta subunits can be outside of the pore region and associated with alpha subunits comprising the pore region. They can also contribute to the external mouth of the pore region.

The term “authentic” or wild-type” or “native” nucleic acid sequences refer to the wild-type nucleic acid sequences contained in the Tables herein as well as splice variants and other nucleic acid sequences generally known in the art.

The term “authentic” or “wild-type” or “native” polypeptides refers to the polypeptide encoded by the genes and nucleic acid sequence contained in the Tables.

The term “modified enhance receptor nuclear acid sequence” or “optimized nucleic acid sequence” refers to a nucleic acid sequence which contains one or mutation that, particularly those that affect (inhibit or enhance) gene activity in recombinant host cells, and most especially oocytes or human cells such as HEK-293 cells. Particularly, these mutations include those that affect gating by the resultant ion channel containing the mutated subunit sequence. The ion channel may comprise such mutations in one or several of the three subunits that constitute the particular ion channel. The modified nucleic acid sequence for example may contain substitution mutations in one subunit that affect (impair) gating function or defective surface expression. The invention embraces the use of other mutated gene sequences, i.e., splice variants, those containing deletions or additions, chimeras of the subject sequences and the like. Further, the invention may use sequences which may be modified to introduce host cell preferred codons, particularly amphibian or human host cell preferred codons.

The term receptor or ion channel protein or transporter or fragment thereof, or a nucleic acid encoding a particular taste receptor or ion channel or transporter or a fragment thereof according to the invention refers to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by the wild-type nucleic acid or amino acid sequence of the taste protein, e.g., proteins encoded by the gene nucleic acid sequences contained in the Tables herein as well as fragments thereof, and conservatively modified variants thereof; (3) polypeptides encoded by nucleic acid sequences which specifically hybridize under stringent hybridization conditions to an anti-sense strand corresponding to a nucleic acid sequence encoding a gene encoded by one of said genes, and conservatively modified variants thereof; (4) have a nucleic acid sequence that has greater than about 60% sequence identity, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to a nucleic acid, e.g., those disclosed herein.

A putative salty or other taste specific gene or polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. Typically these genes will encode proteins that have ion channel activity, i.e., they are permeable to sodium or lithium.

By “determining the functional effect” or “determining the effect on the cell” is meant assaying the effect of a compound that increases or decreases a parameter that is indirectly or directly under the influence of a taste gene, preferably salty taste gene identified herein e.g., functional, physical, phenotypic, and chemical effects. Such functional effects include, but are not limited to, changes in ion flux, membrane potential, current amplitude, and voltage gating, a as well as other biological effects such as changes in gene expression of any marker genes, and the like. The ion flux can include any ion that passes through the channel, e.g., sodium or lithium, and analogs thereof such as radioisotopes. Such functional effects can be measured by any means known to those skilled in the art, e.g., patch clamping, using voltage-sensitive dyes, or by measuring changes in parameters such as spectroscopic characteristics (e.g., fluorescence, absorbance, refractive index), hydrodynamic (e.g., shape), chromatographic, or solubility properties.

“Inhibitors,” “activators,” and “modulators” of the subject taste cell expressed polynucleotide and polypeptide sequences are used to refer to activating, inhibitory, or modulating molecules identified using in vitro and in vivo assays of these polynucleotide and polypeptide sequences. Inhibitors are compounds that, e.g., bind to, partially or totally block activity, decrease, prevent, delay activation, inactivate, desensitize, or down regulate the activity or expression of these taste specific proteins, e.g., antagonists. “Activators” are compounds that increase, open, activate, facilitate, enhance activation, sensitize, agonize, or up regulate protein activity. Inhibitors, activators, or modulators also include genetically modified versions of the subject taste cell specific proteins, e.g., versions with altered activity, as well as naturally occurring and synthetic ligands, antagonists, agonists, peptides, cyclic peptides, nucleic acids, antibodies, antisense molecules, siRNA, ribozymes, small organic molecules and the like. Such assays for inhibitors and activators include, e.g., expressing the subject taste cell specific protein in vitro, in cells, cell extracts, or cell membranes, applying putative modulator compounds, and then determining the functional effects on activity, as described above.

Samples or assays comprising the proteins encoded by genes identified herein that are treated with a potential activator, inhibitor, or modulator are compared to control samples without the inhibitor, activator, or modulator to examine the extent of activation or migration modulation. Control samples (untreated with inhibitors) are assigned a relative protein activity value of 100%. Inhibition of an ion channel is achieved when the activity value relative to the control is about 80%, preferably 50%, more preferably 25-0%. Activation of an ion channel is achieved when the activity value relative to the control (untreated with activators) is 110%, more preferably 150%, more preferably 200-500% (i.e., two to five fold higher relative to the control), more preferably 1000-3000% or higher.

The term “test compound” or “drug candidate” or “modulator” or grammatical equivalents as used herein describes any molecule, either naturally occurring or synthetic compound, preferably a small molecule, or a protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, siRNA, oligonucleotide, ribozyme, etc., to be tested for the capacity to modulate cold sensation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., a gene or sequence contained in the Tables herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nucl. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci., USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., transmembrane domains, pore domains, and cytoplasmic tail domains. Domains are portions of a polypeptide that form a compact unit of the polypeptide and are typically 15 to 350 amino acids long. Exemplary domains include extracellular domains, transmembrane domains, and cytoplasmic domains. Typical domains are made up of sections of lesser organization such as stretches of .beta.-sheet and .alpha.-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include ^{32P}, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1.×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

The term antibody, as used herein, also includes antibody fragments either produced by the modification of whole antibodies, or those synthesized de novo using recombinant DNA methodologies (e.g., single chain Fv), chimeric, humanized or those identified using phage display libraries (see, e.g., McCafferty et al., Nature 348:552-554 (1990)) For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988) and Harlow & Lane, Using Antibodies, A Laboratory Manual (1999); and Goding, Monoclonal Antibodies: Principles and Practice (2d ed. 1986)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to proteins, polymorphic variants, alleles, orthologs, and conservatively modified variants, or splice variants, or portions thereof, can be selected to obtain only those polyclonal antibodies that are specifically immunoreactive with gene products encoding proteins identified in this application and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

Recombinant Expression of Taste (Salty) Gene Identified Herein

To obtain high level expression of a cloned gene, such as those cDNAs encoding the subject genes, one typically subclones the gene into an expression vector that contains a strong promoter to direct transcription, a transcription/translation terminator, and if for a nucleic acid encoding a protein, a ribosome binding site for translational initiation. Suitable eukaryotic and prokaryotic promoters are well known in the art and described, e.g., in Sambrook et al., and Ausubel et al., supra. For example, bacterial expression systems for expressing the taste specific protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. For example, retroviral expression systems may be used in the present invention. As described infra, the subject putative salty taste affecting genes are preferably expressed in human cells such as HEK-293 cells which are widely used for high throughput screening.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence encoding the identified gene and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

The vectors used in the invention may include a regulatable promoter, e.g., tet-regulated systems and the RU-486 system (see, e.g., Gossen & Bujard, Proc. Nat'l Acad. Sci. USA 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with an encoding sequence under the direction of the polyhedrin promoter or other strong baculovirus promoters.

The elements that are typically included in expression vectors also include a replicon that functions in the particular host cell. In the case of E. coli, the vector may contain a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods may be used to produce bacterial, mammalian, yeast or insect cell lines that express large quantities of the desired taste specific protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983). Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the gene.

After the expression vector is introduced into the cells, the transfected cells are cultured under conditions favoring expression of the gene. In some instances, such polypeptides may be recovered from the culture using standard techniques identified below.

Assays for Modulators of Putative Taste Cell Specific Gene Products Identified Herein

Modulation of a putative taste cell specific protein, can be assessed using a variety of in vitro and in vivo assays, including cell-based models as described above. Such assays can be used to test for inhibitors and activators of the protein or fragments thereof, and, consequently, inhibitors and activators thereof. Such modulators are potentially useful in medications or as flavorings to modulate salty or other taste modalities or taste in general or for usage as potential therapeutics for modulating a taste cell related function or phenotype involving one or several of the identified taste cell specific genes reported herein.

Assays using cells expressing the subject taste specific proteins, either recombinant or naturally occurring, can be performed using a variety of assays, in vitro, in vivo, and ex vivo, as described herein. To identify molecules capable of modulating activity thereof, assays are performed to detect the effect of various candidate modulators on activity preferably expressed in a cell.

The channel activity of ion channel proteins in particular can be assayed using a variety of assays to measure changes in ion fluxes including patch clamp techniques, measurement of whole cell currents, radiolabeled ion flux assays or a flux assay coupled to atomic absorption spectroscopy, and fluorescence assays using voltage-sensitive dyes or lithium or sodium sensitive dyes (see, e.g., Vestergarrd-Bogind et al., J. Membrane Biol. 88:67-75 (1988); Daniel et al., J. Pharmacol. Meth. 25:185-193 (1991); Hoevinsky et al., J. Membrane Biol. 137:59-70 (1994)). For example, a nucleic acid encoding an ion channel protein or homolog thereof can be injected into Xenopus oocytes or transfected into mammalian cells, preferably human cells such as HEK-293 cells. Channel activity can then be assessed by measuring changes in membrane polarization, i.e., changes in membrane potential.

A preferred means to obtain electrophysiological measurements is by measuring currents using patch clamp techniques, e.g., the “cell-attached” mode, the “inside-out” mode, and the “whole cell” mode (see, e.g., Ackerman et al., New Engl. J. Med. 336:1575-1595, 1997). Whole cell currents can be determined using standard methodology such as that described by Hamil et al., Pflugers. Archiv. 391:185 (1981).

Channel activity is also conveniently assessed by measuring changes in intracellular ion levels, i.e., sodium or lithium. Such methods are exemplified herein. For example, sodium flux can be measured by assessment of the uptake of radiolabeled sodium or by using suitable fluorescent dyes. In a typical microfluorimetry assay, a dye which undergoes a change in fluorescence upon binding a single sodium ion, is loaded into the cytosol of taste cell specific ion channel-expressing cells. Upon exposure to an agonist, an increase in cytosolic sodium is reflected by a change in fluorescence that occurs when sodium is bound.

The activity of the subject taste cell specific polypeptides can in addition to these preferred methods also be assessed using a variety of other in vitro and in vivo assays to determine functional, chemical, and physical effects, e.g., measuring the binding thereof to other molecules, including peptides, small organic molecules, and lipids; measuring protein and/or RNA levels, or measuring other aspects of the subject polypeptides, e.g., transcription levels, or physiological changes that affects the taste cell specific protein's activity. When the functional consequences are determined using intact cells or animals, one can also measure a variety of effects such as changes in cell growth or pH changes or changes in intracellular second messengers such as IP3, cGMP, or cAMP, or components or regulators of the phospholipase C signaling pathway. Such assays can be used to test for both activators and inhibitors of KCNB proteins. Modulators thus identified are useful for, e.g., many diagnostic and therapeutic applications.

In Vitro Assays

Assays to identify compounds with modulating activity on the subject genes are preferably performed in vitro. The assays herein preferably use full length protein according to the invention or a variant thereof. This protein can optionally be fused to a heterologous protein to form a chimera. In the assays exemplified herein, cells which express the full-length polypeptide are preferably used in high throughput assays are used to identify compounds that modulate gene function. Alternatively, purified recombinant or naturally occurring protein can be used in the in vitro methods of the invention. In addition to purified protein or fragment thereof, the recombinant or naturally occurring taste cell protein can be part of a cellular lysate or a cell membrane. As described below, the binding assay can be either solid state or soluble. Preferably, the protein, fragment thereof or membrane is bound to a solid support, either covalently or non-covalently. Often, the in vitro assays of the invention are ligand binding or ligand affinity assays, either non-competitive or competitive (with known extracellular ligands such as menthol). These in vitro assays include measuring changes in spectroscopic (e.g., fluorescence, absorbance, refractive index), hydrodynamic (e.g., shape), chromatographic, or solubility properties for the protein.

Preferably, a high throughput binding assay is performed in which the protein is contacted with a potential modulator and incubated for a suitable amount of time. A wide variety of modulators can be used, as described below, including small organic molecules, peptides, antibodies, and ligand analogs. A wide variety of assays can be used to identify modulator binding, including labeled protein-protein binding assays, electrophoretic mobility shifts, immunoassays, enzymatic assays such as phosphorylation assays, and the like. In some cases, the binding of the candidate modulator is determined through the use of competitive binding assays, where interference with binding of a known ligand is measured in the presence of a potential modulator. In such assays, the known ligand is bound first, and then the desired compound i.e., putative enhancer is added. After the particular protein is washed, interference with binding, either of the potential modulator or of the known ligand, is determined. Often, either the potential modulator or the known ligand is labeled.

In addition, high throughput functional genomics assays can also be used to identify modulators of cold sensation by identifying compounds that disrupt protein interactions between the taste specific polypeptide and other proteins to which it binds. Such assays can, e.g., monitor changes in cell surface marker expression, changes in intracellular calcium, or changes in membrane currents using either cell lines or primary cells. Typically, the cells are contacted with a cDNA or a random peptide library (encoded by nucleic acids). The cDNA library can comprise sense, antisense, full length, and truncated cDNAs. The peptide library is encoded by nucleic acids. The effect of the cDNA or peptide library on the phenotype of the cells is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the protein encoded by a cDNA according to the invention can be isolated using a yeast two-hybrid system, mammalian two hybrid system, or phage display screen, etc. Targets so identified can be further used as bait in these assays to identify additional components that may interact with the particular ion channel, receptor or transporter protein which members are also targets for drug development (see, e.g., Fields et al., Nature 340:245 (1989); Vasavada et al., Proc. Nat'l Acad. Sci. USA 88:10686 (1991); Fearon et al., Proc. Nat'l Acad. Sci. USA 89:7958 (1992); Dang et al., Mol. Cell. Biol. 11:954 (1991); Chien et al., Proc. Nat'l Acad. Sci. USA 9578 (1991); and U.S. Pat. Nos. 5,283,173, 5,667,973, 5,468,614, 5,525,490, and 5,637,463).

Cell-Based In Vivo Assays

In preferred embodiments, wild-type and mutant taste cell specific proteins are expressed in a cell, and functional, e.g., physical and chemical or phenotypic, changes are assayed to identify modulators that modulate function or which restore the function of mutant genes, e.g., those having impaired gating function. Cells expressing proteins can also be used in binding assays. Any suitable functional effect can be measured, as described herein. For example, changes in membrane potential, changes in intracellular lithium or sodium levels, and ligand binding are all suitable assays to identify potential modulators using a cell based system. Suitable cells for such cell based assays include both primary cells and recombinant cell lines engineered to express a protein. The subject taste cell specific proteins therefore can be naturally occurring or recombinant. Also, as described above, fragments of these proteins or chimeras with ion channel activity can be used in cell based assays. For example, a transmembrane domain of a ion channel or GPCR or transporter gene according to the invention can be fused to a cytoplasmic domain of a heterologous protein, preferably a heterologous ion channel protein. Such a chimeric protein would have ion channel activity and could be used in cell based assays of the invention. In another embodiment, a domain of the taste cell specific protein, such as the extracellular or cytoplasmic domain, is used in the cell-based assays of the invention.

In another embodiment, cellular polypeptide levels of the particular target taste polypeptide can be determined by measuring the level of protein or mRNA. The level of protein or proteins related to ion channel activation are measured using immunoassays such as western blotting, ELISA and the like with an antibody that selectively binds to a polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, protein expression can be measured using a reporter gene system. Such a system can be devised using a promoter of the target gene operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

In another embodiment, a functional effect related to signal transduction can be measured. An activated or inhibited ion channel or GPCR or transporter will potentially alter the properties of target enzymes, second messengers, channels, and other effector proteins. The examples include the activation of phospholipase C and other signaling systems. Downstream consequences can also be examined such as generation of diacyl glycerol and IP3 by phospholipase C.

Assays for ion channel activity include cells that are loaded with ion or voltage sensitive dyes to report activity, e.g., by observing sodium influx or intracellular sodium release. Assays for determining activity of such receptors can also use known agonists and antagonists for these receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. Radiolabeled ion flux assays or a flux assay coupled to atomic absorption spectroscopy can also be used.

Animal Models

Animal models also find potential use in screening for modulators of gene activity. Similarly, transgenic animal technology including siRNA and gene knockout technology, for example as a result of homologous recombination with an appropriate gene targeting vector, or gene overexpression, will result in the absence or increased expression of the target protein. The same technology can also be applied to make knock-out cells. When desired, tissue-specific expression or knockout of the target gene may be necessary. Transgenic animals generated by such methods find use as animal models of responses related to the gene target. For example such animals expressing a gene or genes according to the invention may be used to derive supertaster phenotypes such as for use in screening of chemical and biological toxins, rancid/spoiled/contaminated foods, and beverages or for screening for therapeutic compounds that modulate taste stem cell differentiation.

Knock-out cells and transgenic mice can be made by insertion of a marker gene or other heterologous gene into an endogenous gene site in the mouse genome via homologous recombination. Such mice can also be made by substituting an endogenous gene with a mutated version of the target gene, or by mutating an endogenous gene, e.g., by exposure to known mutagens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual (1988) and Teratocarcinomas and Embryonic Stem Cells: A Practical Approach (Robertson, ed., 1987).

Candidate Modulators

The compounds tested as modulators of the putative taste related proteins or other non-taste related functions and phenotypes involving taste cells can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of a protein. Typically, test compounds will be small organic molecules, peptides, lipids, and lipid analogs. In one embodiment, the compound is a menthol analog, either naturally occurring or synthetic.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md.). C. Solid State and Soluble High Throughput Assays

Additionally soluble assays can be effected using a target taste specific protein, or a cell or tissue expressing a target taste protein disclosed herein, either naturally occurring or recombinant. Still alternatively, solid phase based in vitro assays in a high throughput format can be effected, where the protein or fragment thereof, such as the cytoplasmic domain, is attached to a solid phase substrate. Any one of the assays described herein can be adapted for high throughput screening, e.g., ligand binding, calcium flux, change in membrane potential, etc.

In the high throughput assays of the invention, either soluble or solid state, it is possible to screen several thousand different modulators or ligands in a single day. This methodology can be used for assaying proteins in vitro, or for cell-based or membrane-based assays comprising a protein from a gene identified in this application. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immunol. Meth. 102:259-274 (1987) (describing synthesis of solid phase components on pins); Frank & Doring, Tetrahedron 44:6031-6040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753-759 (1996) (all describing arrays of biopolymers fixed to solid substrates). Non-chemical approaches for fixing tag binders to substrates include other common methods, such as heat, cross-linking by UV radiation, and the like.

Having described the invention supra, the examples provided infra further illustrate some preferred embodiments of the invention. These examples are provided only for purposes of illustration and should not be construed as limiting the subject invention.

### PRACTICAL APPLICATIONS OF THE INVENTION

Compounds which modulate, preferably enhance the activity of genes identified herein in Tables 1-3 have important implications in modulation of human salty taste and potentially other taste modalities or taste in general. In addition these compounds are potentially useful in therapeutic applications involving other taste cell related functions and phenotypes such as taste cell turnover, digestive diseases, digestive function, regulation of metabolism, regulation of immunity in the oral cavity and/or digestive system and the like.

Compounds which activate taste ion channels in taste papillae on the tongue can be used to enhance salt sensation by promoting Na⁺ transport into taste bud cells. This has obvious consumer applications in improving the taste and palatability of low salt foods and beverages.

In addition the genes and gene products herein can be used as markers for identifying, isolating or enriching specific taste cell types or lineages including sweet, bitter, umami, sour, salt, fat, metallic et al.

Further the genes and gene products specific to taste cells identified herein can be used to identify compounds that modulate apoptosis of taste cells, modulate transcription factors that control taste receptor expression, modulate bitter receptor expression e.g., to alleviate the off-taste of some vegetables, medicines, coffee, and the like; modulate autocrine/paracrine modulation of taste cell development, prolong taste bud lifetime, yield supertaster animal phenotypes for use in screening such as for bioterrorism or animals for use in screening for compounds that induce the activation and differentiation of stem cells into taste cells in vivo.

In addition the subject genes and gene products and cells which express may be used to identify ancillary taste receptors or primary taste receptors such as fat or metallic taste cells.

Also the subject genes, gene products and cells which express same can be used in screens to identify compounds that affect digestive function such s gastric motility, food detection, food absorption or the production of digestive fluids, peptides, hormones or enzymes such as GLP-1 (glucagon-like peptide 1), GIP (glucose-dependent insulinotrophic polypeptide), pepsin, secretin, amylase, saliva, et al.

Also the subject genes, gene products and cells which express same may be used to screen for compounds that affect trafficking of taste receptors to and from the apical membrane/taste pore region to enhance or repress general or specific tastes, regulation of taste cell action potential firing frequency/membrane potential to control the intensity of general or specific tastes, regulation of neurotransmitter release to afferent nerve to control the intensity of general or specific taste, and autocrine/paracrine modulation of taste receptor function.

Further the subject genes, gene products and cells which express same can be used to identify compounds that regenerate taste cells such as in geriatric individuals or patients with cancer, chemotherapy radiation, injury or surgery affecting taste, drug-induced dysgeusia, ageusia, and for alleviating taste bud loss.

Still further the subject genes and gene products and cells which express same can be used to screen for compounds that affect oral hygiene, halitosis, detoxification of noxious substances in the oral cavity, and neutralization/elimination of bacteria, viruses, and other immunogens in the saliva/mouth or digestive tract.

Yet additionally the subject genes, gene products and cells which express same can be used in screens to identify compounds that affect saliva production and composition and treatment of dry mouth in conditions such as xerostomia (as for example in Sjogren's syndrome) and autoimmune or inflammatory gastrointestinal diseases such as IBD, ulcerative colitis, and diverticulitis and cancers affecting the oral cavity and digestive tract.

The following examples were effected using the materials and methods described supra. These examples are put forth so as to provide those of ordinary skill in the art with a complete disclosure and description of how to make and use the subject invention, and are not intended to limit the scope of what is regarded as the invention.

### EXAMPLES

### Example 1

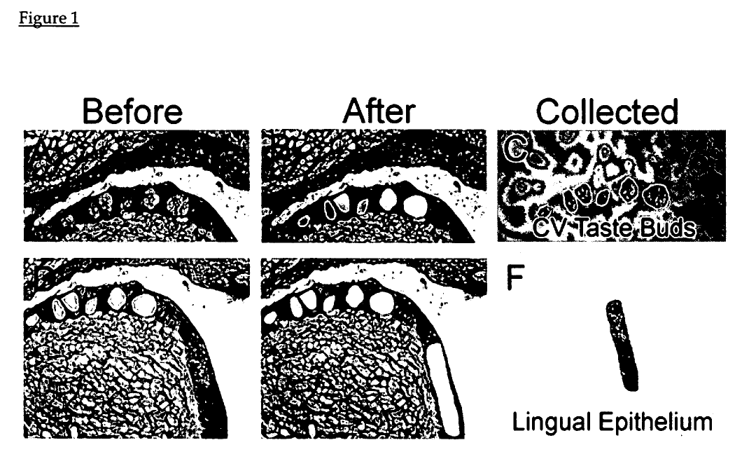

This experiment, the results of which are contained in FIG. 1, is an example of LCM on human taste tissue. A-C: Circumvallate (CV) taste bud collection. D-E: Lingual epithelium collection. Left column shows sections before LCM. Middle column shows sections after LCM. Right column shows collected and isolated CV taste buds (5-10 cells per circular taste bud) and lingual epithelial tissue. Taste bud regions previously collected by LCM are also visible in D and E.

### Example 2

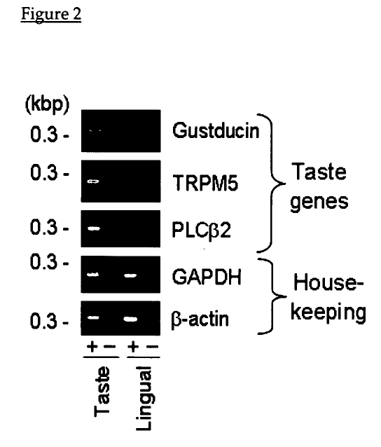

This example, the results of which are contained in FIG. 2, is an example of PCR quality control of human taste and lingual cells. Taste cells, but not lingual cells, specifically express the taste-specific markers gustducin, TRPM5, and PLCβ2. By contrast, both taste and lingual cells express the ubiquitous housekeeping genes GAPDH and β-actin, indicating that taste and lingual cell RNA is intact and of high quality.

### Example 3

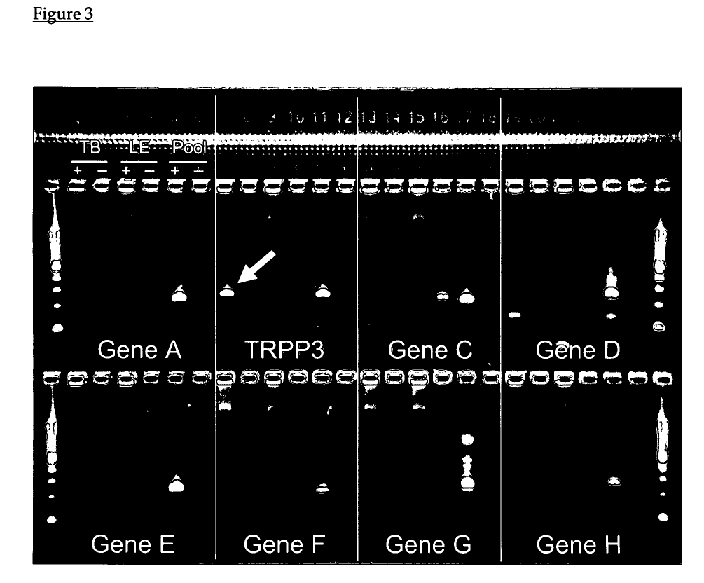

This experiment, the results of which are in FIG. 3, is an example of high-throughput PCR to identify taste-specific ion channels.

Eight different ion channels are shown. For each channel, there are 6 lanes. For each channel, the left two lanes show PCR of human CV taste buds (TB) (+ indicates with reverse transcriptase and − indicates a negative control without reverse transcriptase), middle two lanes show PCR of human lingual epithelium (LE), and right two lanes show PCR with a positive control (denoted pool) to demonstrate that the PCR primers generate a product under the PCR cycling conditions. Note that all 8 ion channels yield a PCR product of the appropriate size in the positive control (pool) but only TRPP3 (also known as PKD2L1) yields a product of the appropriate size in taste buds and not lingual epithelium (yellow arrow). Thus, TRPP3/PKD2L1 is a taste-specific gene.

### Example 4

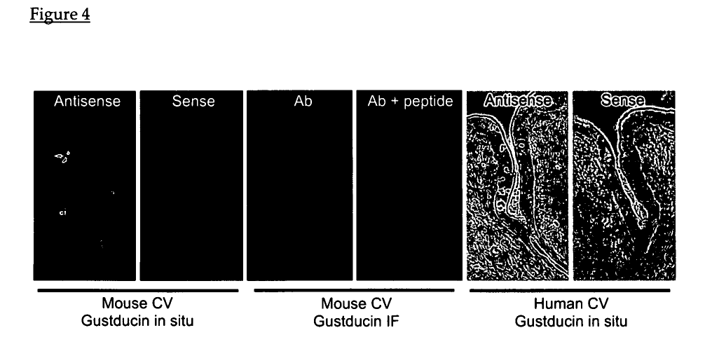

This experiment, the results of which are contained in FIG. 4, is an example of in situ hybridization and immunohistochemistry histological methods to visualize expression of the known taste gene gustducin in mouse or human taste tissue sections. Left images show mouse CV taste buds labeled with the taste-specific G-protein gustducin mouse antisense RNA (green) but not the negative control mouse sense RNA. Middle images show mouse CV taste buds labeled with gustducin using a commercial antibody (red) but not with the negative control (antibody pre-incubated with antigenic peptide). Right images show human CV taste buds labeled with the taste-specific G-protein gustducin human antisense (dark blue) RNA but not the negative control human sense RNA.

### Example 5

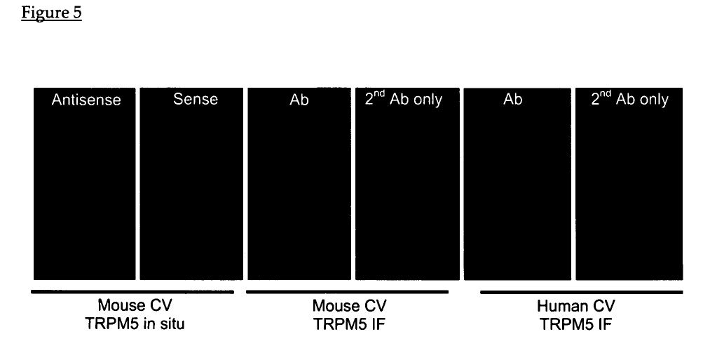

This experiment, the results of which are contained in FIG. 5, is an example of in situ hybridization and immunohistochemistry histological methods to visualize TRPM5 taste gene expression in mouse or human taste tissue sections. Left images show mouse CV taste buds labeled with the taste-specific ion channel TRPM5 mouse antisense RNA (green) but not the negative control mouse sense RNA. Middle images show mouse CV taste buds labeled with TRPM5 using an antibody developed at Senomyx (red) but not with the negative control secondary antibody only. Right images show human CV taste buds labeled with TRPM5 using an antibody developed at Senomyx (red) but not with the negative control secondary antibody only.

### Example 6

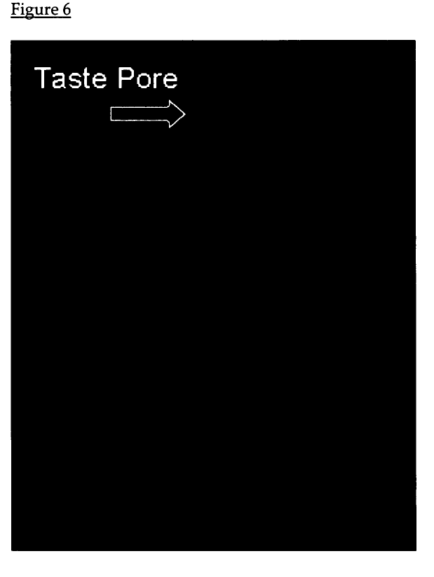

This experiment, the results of which are contained in FIG. 6, is an example of immunohistochemistry to visualize TRPM5 taste gene expression in mouse taste tissue. Image shows mouse circumvallate (CV) taste bud labeled with TRPM5 using an antibody developed at Senomyx. TRPM5 is polarized to the apical taste pore region facing the saliva.

### Example 7

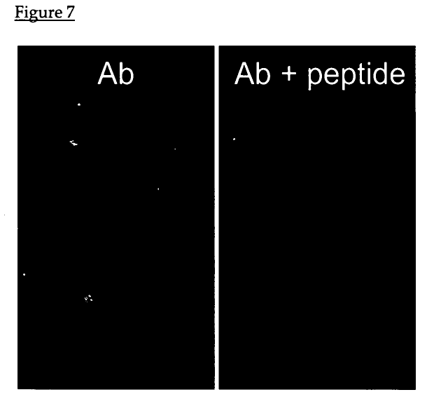

This experiment, the results of which are contained in FIG. 7, is an example of immunohistochemistry histological methods to visualize SCN3A/Nav1.3 sodium channel gene expression in mouse taste tissue. Left image shows mouse CV taste buds labeled with a commercial antibody against SCN3A/Nav1.3 but not with the negative control (antibody pre-incubated with antigenic peptide; right image; Ab+ peptide). This result confirms that SCN3A/Nav1.3 is a taste-specific gene. SCN3A/Nav1.3 was identified as a taste-specific gene by both gene chip and PCR approaches.

### Example 8

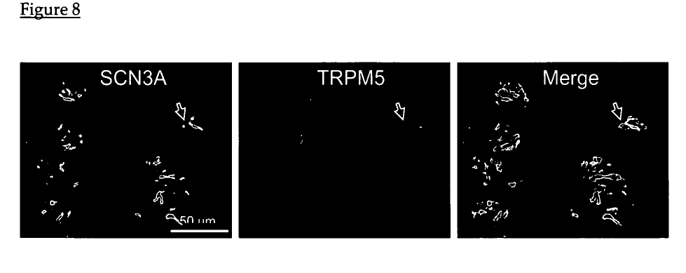

This experiment, the results of which are contained in FIG. 8, is an example of double labeling immunohistochemistry illustrating coexpression of SCN3A and TRPM5 in the same taste cells. SCN3A (left; green) is detectable in cells expressing TRPM5 (middle; red). Overlay of SCN3A and TRPM5 signals is depicted in yellow (right). Since TRPM5 is expressed in cells responsible for sweet, bitter, and umami taste, these data suggest that SCN3A functions to modulate the function of sweet, bitter, and/or umami cells.

SCN3A is expressed in TRPM5 cells responsible for sweet, bitter, and umami taste sensation. Therefore, compounds that modulate SCN3A function could be used to enhance or block sweet, bitter, and/or umami taste.

### Example 9

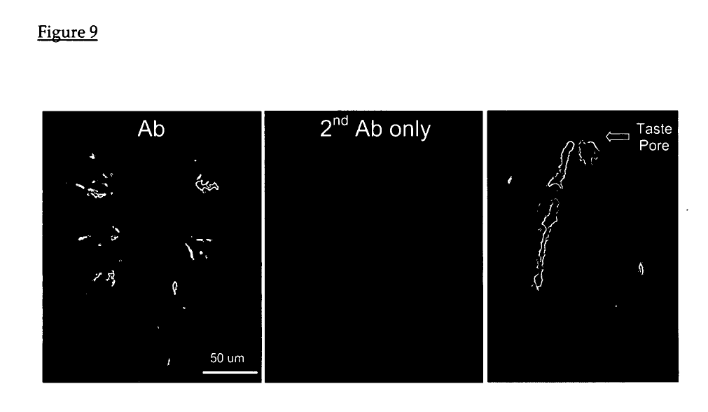

This experiment, the results of which are contained in FIG. 9, is an example of immunohistochemistry to visualize PKD2L1 taste gene expression in mouse taste tissue. Left image shows mouse circumvallate (CV) taste buds labeled with PKD2L1 using a commercial antibody. Middle image shows lack of labeling in an adjacent section incubated with no PKD2L1 primary antibody (negative control secondary antibody only; 2^{nd} Ab only). Right image shows zoom of a single mouse CV taste bud illustrating polarization of PKD2L1 to the apical taste pore region facing the saliva.

### Example 10

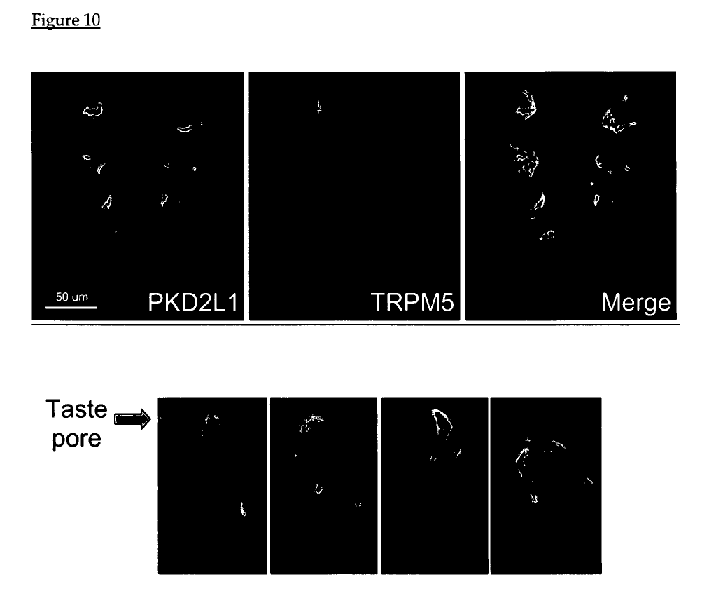

This experiment, the results of which are contained in FIG. 10, is an example of double labeling immunohistochemistry illustrating PKD2L1 and TRPM5 expression in different taste cells. Left image shows mouse CV taste buds labeled with a commercial antibody against PKD2L1 (green); middle image shows labeling with TRPM5 using an antibody developed at Senomyx (red); right image shows merge of PKD2L1 and TRPM5 labels. Bottom 4 images show individual taste buds at high magnification—note that PKD2L1 does not colocalize with TRPM5 (labels are expressed in separate cell types), indicating that PKD2L1 is not expressed in sweet, bitter, and/or umami cells (labeled with TRPM5).

This result confirms that PKD2L1 is a taste-specific gene. PKD2L1 was identified as a taste-specific gene by both gene chip and PCR approaches. PKD2L1 is not expressed in TRPM5 cells responsible for sweet, bitter, and umami taste sensation. Recent reports indicate that PKD2L1/PKD1L3 functions as a sour receptor and that PKD2L1/PKD1L3 is a marker for sour cells (Ishimara et al PNAS 103(33): 12569-12574, 2006; Huang et al Nature 434: 225-229, 2006). Therefore, compounds that modulate PKD2L1/PKD1L3 function could be used to enhance or block sour taste.

### Example 11

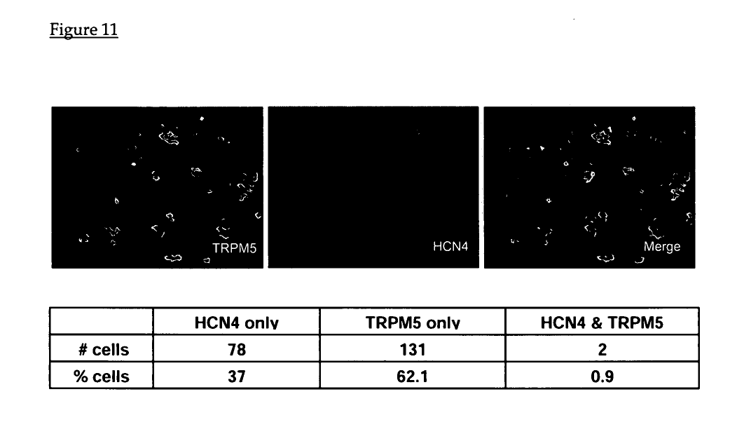

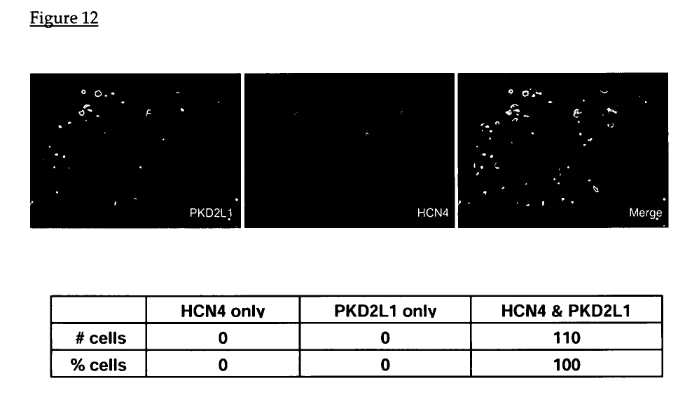

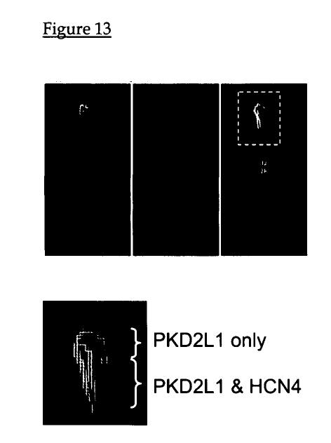

Additional histology experiments were conducted to determine if the hyperpolarization and cyclic-nucleotide gated cation channel HCN4, identified as a taste specific gene in mouse CV papilla by PCR screening, colocalized with TRPM5 (a marker of sweet, bitter, and umami cells), PKD2L1 (a marker of sour cells), or a unique cell type (a putative salt cell). Using antibodies specific to HCN4, it was determined that HCN4 was not expressed in TRPM5 cells (See FIG. 11) but was expressed in PKD2L1 cells (see FIG. 12). As shown in FIG. 11, double labeling immunochemistry was used to visualize HCN4 and TRPM5 in separate mouse CV cells. TRPM5 (left, green) is present in cells not expressing HCN4 (middle, red). The merge of TRPM5 and HCN4 labels is depicted on the right. Since TRPM5 is expressed in cells responsible for sweet, bitter, and umami taste, the data in this figure indicates that HCN4 does not modulate sweet, bitter, or umami taste. The number and percentage of cells expressing HCN4 only, TRPM5 only, or HCN4 and TRPM5 are listed in the table in FIG. 11. FIG. 12 contains the results of another double labeling immunochemistry experiment used to visualize HCN4 and PKD2L1 in the same mouse CV cells. PKD2L1 (left, green) is present in cells expressing HCN4 (middle, red). The merge of PKD2L1 and HCN4 labels is depicted on the right; the yellow color indicates PKD2L1 cells that also express HCN4. Since PKD2L1 is expressed in cells responsible for sour taste, the data therein indicate that HCN4 functions to modulate sour taste and may be used in assays to identify sour taste modulatory compounds. The number and percentage of cells expressing HCN4 only, PKD2L1 only, or HCN4, and PKD2L1 are listed in the table in FIG. 12. HCN4 cells (red) not colocalizing with PKD2L1 cells (green) in the merged image represent cells sloughed off into the CV clefts and are not cells contained in taste buds; therefore, these cells were not counted. As shown by the experimental results contained in FIG. 13, HCN4 was not present in microvilli projecting into the taste pore. Specifically, the double labeling immunochemistry experiment contained in FIG. 13 demonstrates HCN4 exclusion from the taste pore in mouse CV taste cells. [Therein PKD2L1 (left, green), HCN4 (middle, red), and the merge of PKD2L1 and HCN4 (right, yellow).] The same figure shows also that PKD2L1 extends to the tips of taste receptor cells (top of images), whereas HCN4 is excluded from the taste pore region. PKD2L1 and HCN4 colocalize in the cell body, but only PKD2L1 is expressed in the apical membrane. The bottom image shows the zoom of area outlined by the dotted box in the merged image.

Results

Table 1: Summary of mouse taste gene expression from Affymetrix 430 2.0 microarrays/gene chips. Gene chip experiments were conducted on 5 paired mouse CV taste and lingual samples and analyzed using GeneSpring GX v7.3 software (Agilent Technologies). Between 1300 and 2000 CV taste and lingual cells were isolated by LCM and total RNA was isolated and purified. RNA was amplified and hybridized to gene chips. Analyses were performed using 2 separate algorithms: Affymetrix Suite 5 (MAS5) which takes into account the perfect match and mismatch probes on gene chips, and robust multi-chip algorithm (RMA) which takes only into account perfect match probes on gene chips. The table lists genes identified by the following analyses. First, genes were identified and classified as taste-specific genes that fulfilled the following criteria: 1) annotated as encoding transmembrane proteins, 2) expressed greater than or equal to 1.4-fold times higher in taste cells compared to lingual cells by either algorithm, and 3) exhibited raw expression values greater than or equal to 15. These inclusive criteria, as hoped, resulted in the identification of known taste genes, including genes for the sweet, bitter, umami and sour receptors and other genes. Fold changes (ratios of gene expression in taste cells compared to lingual cells), raw counts of gene expression in taste cells, and accession numbers for taste-specific genes encoding membrane proteins are listed. Second, genes were identified as taste-specific genes by the use of the following specific inclusion criteria.

### Inclusion Criteria:

a) Using Affymetrix MAS5 normalized data

CV taste bud mean expression value ≧50

CV versus LE expression ratio ≧4-fold up

CV versus LE expression ratio p value ≦0.05

Identified 433 genes

b) Using Affymetrix GC-RMA normalized data

CV taste bud mean expression value ≧50

CV versus LE expression ratio ≧5-fold up

CV versus LE expression ratio p value ≦0.2

Identified 137 genes

c) PLUS 419 genes identified from both data sets (MAS5 and RMA)
d) Using GeneSpring clustering analysis of MAS5 Affymetrix data

CV taste bud mean expression value ≧100

CV versus LE expression ratio ≧2-fold up

CV versus LE expression ratio t test+Benjamini-Hochberg multiple testing correction ≦0.05

Identified 77 genes

### TOTAL Number Mouse Taste Bud-Specific Genes=1066

Genes from this Second Analysis Encoding Proteins with Multiple Transmembrane Domains with Little or No Functional Characterization are Listed Below.

TABLE 1
Taste Specific Membrane Proteins Identified
in Mouse Circumvallate Cells

| Fold Change - | | |
|---|---|---|
| Taste vs Lingual | Raw Counts - Taste | Accession # |
| 347.47 | 2683.01 | BB096886 |
| 157.2 | 760.78 | BB490331 |
| 65.79 | 625.34 | AF228681 |
| 62.8 | 590.99 | AF228681 |
| 41.32 | 719.17 | BB332752 |
| 29.98 | 207.3 | BE946786 |
| 9.67 | 78.42 | AI549833 |
| 5.91 | 131.44 | NM_013630 |
| 5.66 | 27.01 | BB309395 |
| 3.44 | 28.14 | BB452274 |
| 1.88 | 162.58 | BI685685 |
| 1.78 | 39.46 | NM_010408 |
| 1.74 | 28.11 | AF411816 |
| 1.6 | 222.43 | NM_053177 |
| 10.51 | 184.14 | BI106777 |
| 8.69 | 456.21 | AI848293 |
| 7.09 | 125.82 | AF247139 |
| 4.81 | 36.46 | AB066608 |
| 4.69 | 39.43 | NM_009785 |
| 2.63 | 25.64 | AV326040 |
| 1.98 | 28.65 | BC025890 |
| 1.83 | 167.27 | NM_007581 |
| 1.58 | 65.27 | AW106929 |
| 140.81 | 2868.82 | NM_008430 |
| 108.23 | 208.54 | NM_008434 |
| 103.25 | 826.69 | NM_019659 |
| 65.08 | 1045.39 | NM_020574 |
| 17.58 | 244.35 | AU043100 |
| 14.19 | 54.67 | NM_013569 |
| 8.21 | 121.98 | BQ174236 |
| 8.15 | 51.69 | BE956674 |
| 6.03 | 38.53 | U31908 |
| 4.01 | 30.77 | NM_021452 |
| 3.29 | 36.21 | BM022687 |
| 3.14 | 278.61 | BG865910 |
| 2.21 | 128.82 | BB131475 |
| 1.62 | 43.13 | NM_010596 |
| 12.46 | 251.92 | X83932 |
| 11.36 | 87.44 | M14537 |
| 5.89 | 389.34 | NM_010585 |
| 3.97 | 28.04 | NM_008165 |
| 3.68 | 521.51 | AF089751 |
| 2.47 | 116.84 | AF112185 |
| 2.2 | 44.65 | NM_013561 |
| 2.14 | 231.99 | NM_011325 |
| 2.02 | 91.59 | NM_080553 |
| 1.77 | 56.08 | BB130399 |
| 40.89 | 138.7 | BQ176424 |
| 33.25 | 494.05 | NM_021050 |
| 31.06 | 488.63 | BB345174 |
| 11.41 | 54.93 | BQ176424 |
| 5.16 | 25.84 | BQ175666 |
| 3.36 | 1902.41 | NM_011696 |
| 3.21 | 160.9 | AA210377 |
| 2.9 | 323.95 | BB398988 |
| 2.74 | 34.37 | NM_010298 |
| 2.01 | 62.89 | BC019528 |
| 1.98 | 71.35 | BB236747 |
| 1.72 | 202.21 | AK009435 |
| 1.6 | 292.58 | BB814844 |
| 208.21 | 5078.83 | AB032010 |
| 26.26 | 1657.23 | BB667469 |
| 12.09 | 20.83 | AV226010 |
| 3.55 | 27.29 | BB428982 |
| 2.86 | 245.96 | BC023108 |
| 2.63 | 1898.7 | NM_008557 |
| 2.16 | 46.51 | AV002675 |
| 253.8 | 5079 | AB032010 |
| 235.8 | 10009 | NM_008935 |
| 160 | 2122 | BB217568 |
| 159 | 4570 | BB807707 |
| 131.2 | 2169 | AV371704 |
| 41.3 | 447 | BC025461 |
| 34.5 | 513 | AW494443 |
| 33.7 | 1657 | BB667469 |
| 7.7 | 5094 | AV169215 |
| 12.1 | 21 | AV226010 |
| 8.7 | 16 | AV339322 |
| 87.7 | 2767 | AK009013 |
| 86.2 | 2115 | BG075363 |
| 32.2 | 977 | BC005618 |
| 20.2 | 442 | AK004359 |
| 18.7 | 371 | BB072896 |
| 15 | 355 | NM_019482 |
| 5.8 | 99 | NM_021610 |
| 2.6 | 157 | BC020100 |
| 2.5 | 1244 | BC026372 |
| 2.3 | 9561 | NM_133655 |
| 77.9 | 700 | AK013379 |
| 11.9 | 139 | BC024534 |
| 11.1 | 101 | NM_138751 |
| 8.9 | 50 | AW910499 |
| 6.6 | 576 | AK004283 |
| 4 | 77 | AV032559 |
| 3.5 | 27 | BB428982 |
| 3.5 | 1889 | BG068678 |
| 3.4 | 170 | NM_019953 |
| 3.2 | 118 | BC019745 |
| 2.3 | 286 | NM_019656 |
| 1.9 | 914 | BB747462 |
| 1.9 | 149 | BB431503 |
| 455.8 | 3499 | NM_031873 |
| 75.8 | 1141 | AV337396 |
| 74.9 | 1933 | BC015254 |
| 69.6 | 1965 | BB543291 |
| 27.9 | 1115 | AV024285 |
| 17.3 | 36 | BB086994 |
| 14.6 | 140 | NM_020501 |
| 14.2 | 95 | NM_010098 |
| 10.4 | 361 | BQ173958 |
| 10.1 | 95 | AK010720 |
| 10 | 561 | D87747 |
| 9.2 | 68 | BF159976 |
| 8.7 | 25 | BB317079 |
| 8.5 | 80 | BB273882 |
| 8.1 | 486 | BB259670 |
| 7.9 | 50 | NM_031867 |
| 7.8 | 240 | BB259283 |
| 7.4 | 59 | BC019649 |
| 7.2 | 320 | NM_007974 |
| 6.5 | 107 | AA987131 |
| 6.5 | 66 | NM_031872 |
| 6.2 | 307 | BB709140 |
| 6.2 | 52 | AK009736 |
| 6 | 54 | BB351248 |
| 5.8 | 121 | AF427498 |
| 4.5 | 246 | BE688087 |
| 4 | 79 | BB548889 |
| 3.3 | 37 | AF190670 |
| 3.1 | 46 | AV025152 |
| 3 | 44 | BC020004 |
| 2.9 | 47 | BC016531 |
| 2.8 | 33 | BC022922 |
| 2.4 | 32 | BB009658 |
| 2.2 | 54 | BQ174642 |
| 2 | 32 | NM_013618 |
| 2 | 42 | BB751088 |
| 128.8 | 669 | NM_026228 |
| 27 | 857 | BF165681 |
| 21.9 | 183 | BM938327 |
| 13.6 | 370 | BG069505 |
| 11.9 | 31 | NM_018824 |
| 11.1 | 609 | AV373598 |
| 10.2 | 377 | NM_015747 |
| 9 | 23 | AK003423 |
| 8.4 | 69 | BM237826 |
| 7.7 | 132 | BB732135 |
| 7.3 | 191 | NM_021551 |
| 6.8 | 131 | BC013442 |
| 6.4 | 49 | BB408147 |
| 6.1 | 271 | BB057781 |
| 5.9 | 66 | AF314957 |
| 5.8 | 100 | AW105741 |
| 5.4 | 88 | NM_008063 |
| 5.2 | 81 | AV274006 |
| 5 | 120 | NM_009201 |
| 4.9 | 47 | NM_016981 |
| 4.4 | 228 | AW555750 |
| 4 | 75 | NM_134420 |
| 3.7 | 138 | BG070700 |
| 3.4 | 108 | BB009879 |
| 3.1 | 119 | BC005744 |
| 2.9 | 27 | AW107751 |
| 2.8 | 314 | NM_021301y |
| 2.7 | 3835 | AI303435 |
| 2.6 | 1893 | BB117951 |
| 2.3 | 103 | BB765719 |
| 2.2 | 50 | BC003222 |
| 2.1 | 56 | BC025599 |
| 2.1 | 379 | BB771765 |
| 2 | 44 | NM_011405 |
| 1.9 | 546 | BB410001 |
| 1.7 | 123 | NM_008202 |
| 1.4 | 108 | AW541300 |
| 1.4 | 717 | NM_008135 |
| 1.4 | 2784 | AV327862 |
| 1.4 | 48 | AK005070 |

| RefSeq | LE | TB | Fold change | p |
|---|---|---|---|---|
| Transcript ID | Mean | Mean | TB vs LE | value |
| NM_011696 | 76.2 | 301.5 | 4.0 | 0.0239 |
| NM_023557 | 3.6 | 24.5 | 6.9 | 0.0616 |
| NM_025791 | 50.3 | 215.5 | 4.3 | 0.0741 |
| NM_019656 | 15.2 | 62.4 | 4.1 | 0.0328 |
| NM 030694 | 16.3 | 85.1 | 5.2 | 0.0002 |
| NM_133681 | 3.3 | 417.1 | 128.3 | 0.0014 |
| NM_023056 | 7.6 | 98.1 | 12.9 | 0.0002 |
| NM_025359 | 4.4 | 701.9 | 158.8 | 0.0031 |
| NM_001025371 | 13.0 | 41.7 | 3.2 | 0.0372 |
| R75193 | 2.2 | 53.2 | 24.2 | 0.1030 |
| NM_029478 | 69.9 | 338.4 | 4.8 | 0.0240 |
| NM_025378 | 31.3 | 126.6 | 4.0 | 0.0235 |
| NM_025326 | 2.3 | 75.2 | 32.9 | 0.0136 |
| NM_028058 | 58.6 | 246.1 | 4.2 | 0.0047 |
| NM_026820 | 2.2 | 15.3 | 6.9 | 0.0624 |
| NM_027533 | 1.9 | 9.1 | 4.8 | 0.2068 |
| NM_146010 | 1.5 | 77.8 | 51.0 | 0.0292 |
| NM_022996 | 69.0 | 364.6 | 5.3 | 0.0300 |
| NM_172608 | 3.1 | 77.0 | 24.8 | 0.0248 |
| NM_001002267 | 506.9 | 2188.5 | 4.3 | 0.0038 |
| NM_028651 | 19.1 | 388.4 | 20.3 | 0.0026 |
| NM_010581 | 21.8 | 64.2 | 2.9 | 0.1864 |
| NM_029662 | 4.4 | 27.1 | 6.2 | 0.0904 |
| NM_133352 | 14.0 | 102.1 | 7.3 | 0.1324 |
| NM_029398 | 1.9 | 10.5 | 5.4 | 0.0176 |
| NM_029529 | 1.9 | 7.7 | 4.1 | 0.0990 |
| NM_027152 | 3.5 | 89.0 | 25.6 | 0.0860 |
| NM_178577 | 14.0 | 58.3 | 4.2 | 0.1165 |
| NM_030174 | 1.7 | 75.5 | 43.4 | 0.0700 |
| NM_001013028 | 2.2 | 67.8 | 31.3 | 0.0410 |
| XM_133786 | 4.0 | 40.8 | 10.1 | 0.0397 |
| XM_888885 | 2.0 | 34.5 | 17.2 | 0.0940 |
| NM_172702 | 2.6 | 18.9 | 7.2 | 0.0927 |
| NM_001033381 | 8.4 | 53.6 | 6.4 | 0.1992 |
| XM_001000944 | 211.4 | 1284.4 | 6.1 | 0.0002 |
| NM_153579 | 1.5 | 9.2 | 6.2 | 0.1845 |
| NM_029862 | 3.7 | 46.0 | 12.5 | 0.0666 |
| XM_355152 | 2.1 | 15.1 | 7.3 | 0.1563 |
| NM_013790 | 5.8 | 278.7 | 48.1 | 0.1461 |
| AK148567 | 1.6 | 36.3 | 22.5 | 0.0185 |
| NM_028975 | 6.9 | 37.3 | 5.4 | 0.1667 |
| XM_988868 | 2.7 | 205.4 | 76.8 | 0.0455 |
| BB389250 | 1.6 | 247.4 | 157.7 | 0.0028 |
| NM_009320 | 2.3 | 11.2 | 4.8 | 0.0134 |
| NM_146073 | 24.1 | 339.4 | 14.1 | 0.0891 |
| NM_145700 | 25.2 | 330.9 | 13.1 | 0.0257 |
| NM_009842 | 69.6 | 566.3 | 8.1 | 0.0024 |
| NM_008851 | 2.1 | 4.6 | 2.2 | 0.0652 |
| NM_031999 | 21.4 | 165.4 | 7.7 | 0.1304 |
| NM_023055 | 9.6 | 64.7 | 6.7 | 0.0382 |
| NM_016969 | 19.7 | 126.7 | 6.4 | 0.0006 |
| XM_488763 | 6.0 | 169.6 | 28.2 | 0.0814 |
| NM_010605 | 1.8 | 61.9 | 35.2 | 0.0510 |
| NM_178656 | 1.9 | 61.9 | 33.1 | 0.1988 |
| XM_128954 | 2.0 | 99.0 | 49.6 | 0.0742 |
| NM_146118 | 24.5 | 88.0 | 3.6 | 0.0270 |
| AI504336 | 1.7 | 4.5 | 2.6 | 0.0494 |
| NM_019760 | 11.7 | 62.5 | 5.3 | 0.0849 |
| NM_134020 | 69.1 | 326.9 | 4.7 | 0.0188 |
| NM_175036 | 11.0 | 116.2 | 10.6 | 0.1016 |
| NM_145398 | 9.9 | 121.3 | 12.3 | 0.1355 |
| NM_009728 | 20.4 | 131.6 | 6.5 | 0.0300 |
| NM_198409 | 4.5 | 613.7 | 136.7 | 0.0086 |
| NM_026281 | 2.3 | 8.9 | 4.0 | 0.0801 |
| NM_178874 | 3.1 | 56.1 | 17.8 | 0.0052 |
| NM_173007 | 2.3 | 43.5 | 19.0 | 0.0013 |
| NM_153550 | 7.5 | 48.2 | 6.5 | 0.0757 |
| NM_172715 | 16.0 | 175.2 | 11.0 | 0.0365 |
| NM_172510 | 1.8 | 9.6 | 5.3 | 0.0027 |
| NM_024249 | 28.7 | 145.2 | 5.1 | 0.0017 |
| BE447661 | 3.2 | 88.5 | 28.1 | 0.1953 |
| NM_001024703 | 3.6 | 57.6 | 15.8 | 0.1510 |
| NM_178642 | 26.8 | 544.5 | 20.3 | 0.0024 |
| NM_133978 | 2.3 | 26.2 | 11.5 | 0.1141 |

TABLE 2
Summary of taste-specific ion channels from PCR screens. Genes yielding
PCR products specifically in human and/or mouse taste cells or genes
yielding PCR products that were more abundant in taste cells than lingual
cells are listed. DNA sequencing confirmed the identity of all taste-
specific genes. Accession numbers for taste-specific genes are listed.
Accession #

NM_006922
NM_000335
NM_014191
NM_002977
NM_018400
NM_020897
NM_007332
NM_002420
NM_020952
NM_014555
NM_016112
NM_181536
NM_018298
NM_000720
NM_201596
NM_006539
NM_006030
NM_002234
NM_004770
NM_004978
NM_004979
NM_012281
NM_002237
NM_000218
NM_004519
NM_000238
NM_030779
NM_012284
NM_002248
NM_002249
NM_000891
NM_002240
NM_002241
NM_018658
NM_002246
NM_005472
NM_000352
NM_130770
NM_182589
NM_180990
NM_000080
NM_007325
NM_175611
NM_000835
NM_020039
NM_018674
NM_053277
NM_017682
NM_001287
NM_000806
NM_022003
NM_024780
NM_173160
NM_020277
NM_181422
NM_181544
XM_909341
NM_018732
NM_021544
NM_001033317
NM_008226
NM_001081192
NM_011644
NM_016984
NM_011706
NM_153417
NM_177781
NM_029772

TABLE 3
Summary of colocalization of taste-specific genes in TRPM5 (sweet,
bitter, umami) and PKD2L1/PKD1L3 (sour) cells in histology experiments
by in situ hybridization and/or immunohistochemistry.

| | | Colocalize | |
|---|---|---|---|
| Accession | Colocalize | PKD2L1/ | |
| # | TRPM5 | PKD1L3 | Notes |
| NM_020277 | + | − | Label sweet, bitter, |
| | | | and umami cells |
| NM_181422 | − | + | Label sour cells |
| NM_181544 | − | + | Label sour cells |
| XM_909341 | + | + | Label most all taste |
| | | | cells |
| NM_018732 | + | − | Label sweet, bitter, |
| | | | and umami cells |
| AI549833 | + | − | Label sweet, bitter, |
| | | | and umami cells |
| NM_001081192 | − | + | Label sour cells |
| AK013379 | + | + | Label sweet, bitter, |
| | | | and umami cells |
| NM_019656 | + | − | Label subset of TRPM5 |
| | | | cells |

As afore-mentioned the taste cell specific genes identified herein and the corresponding gene products and cells which express same e.g., endogenous taste or chemosensory cells and recombinant cells including the taste specific genes recited in Tables 1, 2 and 3, and their orthologs, allelic variants, variants possessing at least 90% sequence identity thereto and/or genes which specifically hybridize thereto under hybridization conditions denied supra may be used in assays to identify taste modulatory compounds as well as in therapeutic screening assays.

For example these taste specific genes, polypeptides and cells expressing same can be used to screen for compounds for treatment of digestive system disorders. These disorders include by way of example conditions affecting digestion such as dyspepsia, autoimmune and inflammatory diseases affecting the digestive system such as ulcerative colitis, inflammatory bowel syndrome, Crohn's syndrome, celiac disease, and precancers and cancers that affect the digestive system such as cancers affecting the salivary glands, taste buds, stomach, pancreas, gall bladder, esophagus, anus or colon.

Also these taste specific genes may be used in screening assays to identify compounds that affect taste cell turnover. It is known that taste cells turnover rapidly (about every couple of weeks). Moreover, many conditions including chemotherapy or radiation treatment, as well as old age may negatively affect the ability of taste cells to develop. The result is a diminished sense of taste which may result in a decreased quality of life in cancer patients or the elderly. This is particularly pronounced in patients with head and neck cancer, esophageal, stomach, lung, or pancreatic cancers. Additionally, this may evolve along with another condition, cachexia or wasting syndrome that combines to reduce the desire to eat. Lack of proper nutrition is a serious cause of morbidity and mortality in cancer patients. The subject taste specific genes contain genes expressed in stem cells suggesting that they are markers of stem cells that are the precursors of and which evolve into taste cells. These genes or cells which express same can be used to identify signals that accelerate taste cell development. These signals which likely comprise cytokine-like receptors present on taste cells likely mediate taste cell development and can be used in screens to identify compounds that induce taste cell differentiation or proliferation. The ligands therefore potentially may be isolated from saliva and may account for the ability of saliva to influence taste function. For example, patients with Sjogren's syndrome (an autoimmune disease that attacks the salivary glands) exhibit altered taste functions. The subject genes and the study of gene expression in the salivary glands by use of gene arrays will facilitate an understanding of these differentiation mechanisms.

The subject taste cell specific genes and corresponding gene products and cells which express these genes may also be used in order to identify potential therapeutics for modulating the immune system of the oral cavity. The oral cavity is populated by normal flora as is the digestive tract. Alterations in normal flora may give rise to conditions such as gingivitis, halitosis, gastric problems and other infections that may result in tooth decay or tooth loss. Included within the taste cell specific genes identified herein are a number of immune system genes. These genes and the corrresponding polypeptides or cells which express same can be used to identify therapeutics for maintaining immune homeostasis in the oral cavity, preventing overgrowth of pathogenic microbia, and for identification of the cell types in the oral cavity that are the key players in maintaining proper oral cavity immunity.

Moreover, the subject taste cell specific genes and the corresponding gene products or cells which express same are useful in screening assays for identifying compounds for treatment of diabetes, eating disorders such as obesity, anorexia, bulimia, and other metabolic disorders. The expression of taste receptors in the digestive system likely represents a comprehensive system that detects food and different types at different places during digestion. Therefore, “sensing” the presence of food or specific types such as carbohydrates, fats, umami foods, salts, should trigger various signals that may regulate the production of molecules that participate in the regulation of digestion such as GIP (glucose-dependent insulinotrophic polypeptide) and GLP-1 (glucagon-like peptide 1) produced by the enteroendocrine cells in the intestine. It is likely that taste receptors on these cells regulate the production of other molecular signals in other cells of the digestive system when triggered. These phenomena may be studied by determining which cells express different receptors and then using gene arrays to study the molecules that these cells produce when activated.

### REFERENCES

All the references cited in this application are incorporated by reference in their entirety herein.

1-125. (canceled)

126. A method for identifying a gene encoding a polypeptide involved in salty taste perception in a mammal comprising:
(i) identifying a set of genes including genes which are expressed in taste cells but which are not expressed in lingual cells and/or genes which are expressed in taste cells at substantially higher levels than in lingual cells;
(ii) of the genes identified in (i) identifying a set of genes which are not expressed in taste cells which express umami, sweet, bitter, or sour taste receptors or markers of these cells (T1Rs or T2Rs, TRPM5, and PKD2L1/PKD1L3); and
(iii) functionally expressing one or more genes identified according to (ii) and determining which of said genes functions as a sodium responsive ion channel or sodium responsive receptor or transporter and thereby identifying this gene or genes as a putative gene that modulates salty taste.

127. The method of claim 127 wherein step (i) comprises the use of laser capture microdissection (LCD) to dissect and purify taste tissues from non-taste tissues.

128. The method of claim 127 wherein step (i) comprises RNA amplification of genes from taste cells and lingual cells and the amplified genes are screened against a gene chip containing a sample of genes specific to the particular mammal from which the taste and lingual tissues are obtained.

129. The method of claim 127 wherein the taste tissues are derived from human or rodent source.

130. The method of claim 127 wherein the gene chips include a set of annotated mammalian genes.

131. The method of claim 127 wherein step (i) comprises high throughput PCR using primers for each ion channel in the human or mammalian genome.

132. The method of claim 127 wherein step (ii) is effected by in situ hybridization using antisense RNA probes specific for the genes identified in step (i) to determine level of expression in taste versus lingual cells.

133. The method of claim 127 wherein step (ii) is effected by use of immunochemical detection using a labeled antibody specific to the protein encoded by gene or genes identified in step (i).

134. The method of claim 127, wherein said identified gene or genes are characteristic of cells which do not express TRPM5 or PKD2L1/PKD1L3.

135. A method for selecting cells which do not express TRPM5 or PKD2L1/PKD1L3, comprising determining whether a cell expresses a gene identified according to the method of claim 127, wherein cells expressing said gene are less likely to express TRPM5 or PKD2L1/PKD1L3.

136. The method of claim 127, wherein said gene is selected from the genes contained in any one of tables 1-3.

137. The method of claim 127, wherein said genes function as a sodium responsive ion channel, and further wherein a fraction of the channel population is open and passing sodium at rest.

138. A method for identifying a gene encoding a polypeptide involved in salty taste perception in a mammal comprising:
(i) identifying a set of genes including genes which are expressed in taste cells but which are not expressed in lingual cells and/or genes which are expressed in taste cells at substantially higher levels than in lingual cells;
(ii) of the genes identified in (i) identifying a set of genes which are not expressed in taste cells which express umami, sweet, bitter, or sour taste receptors or markers of these cells (T1Rs or T2Rs or TRPM5 or PKD2L1/PKD1L3); and
(iii) determining, in a primary neuron which expresses one or more genes identified according to (ii), which of said genes functions as a sodium responsive ion channel or sodium responsive receptor or transporter and thereby identifying this gene or genes as a putative gene that modulates salty taste.

139. The method of claim 138 wherein said genes include those recited in one of Tables 1, 2, or 3 or an ortholog or allelic variant, or a variant that encodes a protein that is at least 90% identical to the protein encoded by said gene or ortholog thereof.

140. An assay for identifying a compound having potential in vivo application for modulating human salty taste comprising the following:
(i) contacting a cell that expresses a gene encoding an ion channel, receptor or transporter identified according to any one of claim 127 or a gene encoding a polypeptide possessing at least 90% sequence identity to the polypeptide encoded thereby with at least one putative enhancer compound;
(ii) assaying sodium conductance, receptor activity or sodium transport in the presence and absence of said putative enhancer; and
(iii) identifying the compound as a potential salty taste enhancer based on whether it increases sodium conductance, the activity of said receptor or sodium transport.

141. The method of claim 140 wherein said gene encodes an ion channel.

142. The method of claim 140 wherein said gene encodes a GPCR.

143. The method of claim 140 wherein said gene is a human or mammalian gene.

144. The method of claim 140 which further includes testing the effect of said compound or a derivative thereof in a human taste test.

145. The method of claim 140 wherein the putative salty taste affecting gene is expressed in an amphibian oocyte.

146. The method of claim 140 wherein the putative salty taste affecting gene is expressed in a mammalian cell.

147. The method of claim 140 wherein said assay is an electrophysiological assay which uses a sodium sensitive dye.

148. The method of claim 147 wherein said assay is a two electrode voltage clamping assay.

149. The method of claim 147 wherein the test cell is a Xenopus oocyte or a mammalian cell.

150. The method of claim 149 wherein said mammalian cell is selected from the group consisting of a HEK293, HEK293T, Swiss3T3, CHO, BHK, NIH3T3, and COS cell.

151. The method of claim 149 wherein the cell is a Xenopus oocyte.

152. The method of claim 147 wherein said assay is a patch clamp assay.

153. The method of claim 140 which uses a membrane potential dye is selected from the group consisting of Molecular Devices Membrane Potential Kit (Cat#R8034), Di-4-ANEPPS (pyridinium, 4-(2-(6-(dibutylamino)-2-naphthalen-yl)ethenyl)-1-(3-sulfopropyl)hydroxide, inner salt, DiSBACC4(2)(bis-(1,2-dibabituric acid)-triethine oxanol), Cc-2-DMPE (Pacific Blue 1,2-dietradecanoyl-sn-glycerol-3phosphoethanolamine, triethylammonium salt) and SBFI-AM (1,3-benzenedicrboxylic acid, 4,4-[1,4,10-trioxa-7,13-diazacylopentadecane-7,13-diylbis(5-methoxy-6,1,2-benzofurandiyl)}bis-tetrakis {(acetyloxy)methyl}ester (Molecular Probes).

154. The method of claim 147 wherein said sodium sensitive dye is sodium green tetraacetate (Molecular Probes) or Na-sensitive Dye Kit (Molecular Devices).

155. The method of claim 127 wherein the assay measures activity by an ion flux assay.

156. The method of claim 155 which uses atomic absorption spectroscopy to detect ion flux.

157. The method of claim 127 wherein the putative salty taste affecting gene is expressed under the control of a regulatable promoter.

158. The method of claim 127 which uses a fluorescence plate reader (FLIPR).

159. The method of claim 127 which uses a voltage imaging plate reader (VIPR) which is used to increase sodium or fluid absorption.

160. The method of claim 140 wherein the identified compound promotes sodium ion transport into taste bud cells.

161. The method of claim 127 which uses a membrane potential dye selected from the group consisting of Molecular Devices Membrane Potential Kit (cat#8034), Di-4-ANEPPS (pyridinium, 4-(2-(6-(dibutylamino)-2-naphthalen-yl)ethenyl)-1-(3-sulfopropyl)-hydroxide, inner salt); DiSBACC4(2)(bis-(1.2-dibarbituric acid)-trimethine oxanol); DiSBAC4(3) (bis-(1,3-dibarbituric acid)-trimethine oxanol); CC-2-DPME (Pacific Blue 1,2-dietradecanoyl-sn-glycerol-3-phosphoethanolamine, triethylammonium salt) and SBFI-AM (1,3-Benzenedicarboxylic acid, 4,4′-[1,4,10-trioxa-7,13-diazacyclopentadecane-7,13-diylbis(5-methoxy-6,1,2-benzofurandiyl)]bis-tetrakis[(acetyloxy)methyl]ester (Molecular Probes).

162. The method of claim 127 which uses a cell that stably expresses said putative salty taste affecting gene.

163. The method of claim 127 which uses a cell that transiently expresses said putative salty taste affecting gene.

164. The method of claim 127 wherein activity is monitored using a sodium sensitive dye.

165. The method of claim 164 wherein said dye is sodium green tetraacetate (Molecular Probes) or Na-sensitive Dye Kit (Molecular Devices).

166. The method of claim 127 wherein activity is assayed in a frog oocyte electrophysiologically by patch clamping or two electrode voltage clamping.

167. The method of claim 127 which uses an automatic imaging instrument.

168. The method of claim 167 wherein said instrument is a fluorescence plate reader (FLIPR).

169. The method of claim 167 wherein said instrument is a voltage imaging plate reader (VIPR).

170. The method of claim 127 wherein said gene is expressed by a cell selected from the group consisting of HEK-293, BHK, CHO, COS, monkey L cell, African green monkey kidney cell, Ltk-cell and an oocyte.

171. The method of claim 127 wherein the screened genes include those contained in any one of Tables 1-3.

172. The method of claim 170 wherein the cell is an HEK-293 cell.

173. An assay for identifying a compound having potential in vivo application for modulating human sweet, bitter or umami taste comprising the following:
(i) contacting a cell that expresses a gene listed in Tables 1-3 present in TRPM5 taste cells with at least one putative enhancer compound;
(ii) assaying sodium conductance, receptor activity or sodium transport in the presence and absence of said putative enhancer; and
(iii) identifying the compound as a potential enhancer for sweet, bitter or umami taste based on whether it increases sodium conductance, the activity of said receptor or sodium transport.

174. The assay of claim 173 wherein the cell is a mammalian cell or a frog oocyte.

175. The assay of claim 173 wherein the mammalian cell is a CHO, Cos, BHK or HEK-293 cell.

176. The assay of claim 175 wherein the cell is a HEK-293 cell.

177. The assay of claim 173 wherein the effect of said compound on sweet taste is confirmed in taste tests.

178. The assay of claim 173 wherein the effect of said compound on umami taste is confirmed in taste tests.

179. The assay of claim 173 wherein the effect of said compound on bitter taste is confirmed in taste tests.

180. The assay of claim 173 wherein the taste tests are effected with human volunteers.

181. An assay for identifying a putative sour taste modulator comprising:
(i) contacting a cell that expresses a gene listed in Tables 1-3 present in PKD2L1/PKD1L3 taste cells and optionally another gene involved in sour taste perception with a compound;
(ii) assaying the effect of said compound on ion transport, ion conductance, or an activity elicited by said ion channel; and
(iii) identifying said compound as a sour taste modulator if it has an effect on ion transport, ion conductance, or an activity elicited by said channel.

182. The assay of claim 181 wherein the ion is sodium.

183. The assay of claim 181 wherein the effect of said compound on sour taste is confirmed in taste tests.

184. The assay of claim 183 wherein said taste tests are effected in human volunteers.

185. The assay of claim 183 which includes the addition of a compound known to elicit a sour taste and the assay screens the effect of said compound on ion transport, ion conductance, or an activity elicited by said ion channel.

186. The assay of claim 183 wherein the cell also expresses PKD2L1/PKD1L3.

187. A method of using a taste specific polypeptide encoded by gene selected from the genes recited in Table 1 or Table 2 or Table 3 or an ortholog or variant thereof that possesses at least 90% sequence identity therewith in a binding or functional assay method that screens for compounds that specifically bind and/or modulate the activity of said taste specific polypeptide and based on said screening assay identifying compounds that putatively modulate at least one of taste cell development, senescence, apoptosis, or taste bud regeneration.

188. The method of claim 183 wherein the screening assay is effected in a transgenic, animal that expresses said gene or in a genetically modified animal that exhibits reduced or eliminated expression of said gene using siRNA or gene knockout approaches.

189. A method of using a taste specific polypeptide encoded by gene selected from the genes recited in Table 1 or Table 2 or Table 3 or an ortholog or variant thereof that possesses at least 90% sequence identity therewith in a binding or functional assay method that screens for compounds that specifically bind and/or modulate the activity of said taste specific polypeptide and based on said screening assay identifying compounds that putatively modulate gastrointestinal function.

190. The method of claim 189 wherein the cells are taste or gastrointestinal cells and the assay screens for compounds that affect one of gastric motility, food sensing, food absorption, or peptide secretion.

191. The method of claim 190 wherein the screened compounds affect secretion of at least one of amylase, GLP-1 (glucagon-like peptide 1), secretin, pepsin, and GIP (glucose-dependent insulinotrophic polypeptide).

192. The method of claim 189 wherein the identified compounds are evaluated in vivo for their effect on taste or gastrointestinal function.

193. The method of claim 192 wherein gastrointestinal function includes nutrient absorption, nutrient sensing, ion transport, peptide or hormone or enzyme secretion, and/or appetite.

194. A method of using a taste specific polypeptide encoded by gene selected from the genes recited in Table 1 or Table 2 or Table 3 or an ortholog or variant thereof that possesses at least 90% sequence identity therewith in a binding or functional assay method that screens for compounds that specifically bind and/or modulate the activity of said taste specific polypeptide and based on said screening assay identifying compounds having potential therapeutic efficacy in treating or preventing a pathological condition involving the digestive system or a digestive organ.

195. The method of claim 194 wherein the condition is functional dyspepsia or another dyspepsia which is ulcer or non-ulcer related.

196. The method of claim 194 wherein the therapeutic compound influences a hormone involved with hunger or digestion.

197. The method of claim 196 wherein the protein is selected from gastrin, secretin, amylase, cholecystokinin, glucose-dependent insulinotrophic polypeptide, glucagon like peptide 1 ghrelin, or leptin.

198. The method of claim 194 wherein the therapeutic compound is useful for treating an inflammatory or autoimmune gastrointestinal disease.

199. The method of claim 198 wherein the disease is selected from celiac disease, inflammatory bowel syndrome, Crohn's disease, Sjogren's syndrome, gastritis, diverticulitis, or ulcerative colitis.

200. The method of claim 194 wherein the therapeutic compound is used to treat or prevent a digestive system or digestive organ associated cancer.

201. The method of claim 200 wherein the cancer affects an organ selected from the colon, small or large intestine, anus, liver, pancreas, gall bladder, esophagus, tongue, salivary gland, taste bud, or stomach cancer or a cancer-associated cachexia.

202. The method of claim 194 wherein the therapeutic compound is used to treat or prevent an appetite related disease or dysfunction.

203. The method of claim 202 wherein the appetite related disease or condition is bulimia or anorexia, or a cachexia associated therewith.

204. The method of claim 202 wherein the therapeutic compound is used to treat or prevent a disorder or disease associated with gastric reflux.

205. The method of claim 204 wherein the disorder or disease is selected from gastroesophageal reflux disease, esophagitis, Barrett's esophagus, or heartburn.

206. A method of using a taste specific polypeptide encoded by gene selected from the genes recited in Table 1 or Table 2 or Table 3 or an ortholog or variant thereof that possesses at least 90% sequence identity therewith in a binding or functional assay method that screens for compounds that specifically bind and/or modulate the activity of said taste specific polypeptide and based on said screening assay identifying compounds useful for modulating the immune system in the oral cavity or gastrointestinal tract.

207. The method of claim 206 which is used to screen for compounds useful for treating gingivitis, halitosis, or neutralizing or inhibiting a noxious substance or microbe therein.

208. A method of using a taste specific polypeptide encoded by gene selected from the genes recited in Table 1 or Table 2 or Table 3 or an ortholog or variant thereof that possesses at least 90% sequence identity therewith in a binding or functional assay method that screens for compounds that specifically bind and/or modulate the activity of said taste specific polypeptide and based on said screening assay identifying
a compound useful for treating one of xerostomia (for example as in Sjogren's syndrome), dysgeusia or ageusia.

209. A method of using a taste specific polypeptide encoded by gene selected from the genes recited in Table 1 or Table 2 or Table 3 or an ortholog or variant thereof that possesses at least 90% sequence identity therewith in a binding or functional assay method that screens for compounds that specifically bind and/or modulate the activity of said taste specific polypeptide and based on said screening assay identifying compounds useful for treating a metabolic disorder involving taste or digestive cell function.

210. The method of claim 209 wherein the metabolic disorder is diabetes or obesity.

211. A method of using a taste specific polypeptide encoded by gene selected from the genes recited in Table 1 or Table 2 or Table 3 or an ortholog or variant thereof that possesses at least 90% sequence identity therewith in a binding or functional assay method that screens for compounds that specifically bind and/or modulate the activity of said taste specific polypeptide and based on said screening assay identifying compounds that affect at least one of trafficking of taste cell receptors, taste cell neurotransmitter release, autocrine/paracrine modulation of taste receptor function, and taste cell action potential firing frequency/membrane potential.

212. A method of using a taste specific polypeptide encoded by gene selected from the genes recited in Table 1 or Table 2 or Table 3 or an ortholog or variant thereof that possesses at least 90% sequence identity therewith in a binding or functional assay method that screens for compounds that specifically bind and/or modulate the activity of said taste specific polypeptide and based on said screening assay identifying compounds potentially useful for treating taste cell loss following injury, cancer, chemotherapy, radiation, injury or surgery, or the result of age-related taste bud loss.

213. An isolated or purified taste or gastrointestinal cell that expresses at least one gene recited in Table 1, 2 or 3.

214. The isolated taste cell of claim 213 which further expresses alpha ENaC, cytokeratin 19, and/or C6orf15.

215. The isolated taste cell of claim 213 which does not express a T1R.

216. The isolated taste cell of claim 213 which does not express a T2R.

217. The isolated taste cell of claim 213 which does not express PKD2L1/PKD1L3 and/or TRPM5.

218. The isolated taste cell of claim 213 which is human.

219. The isolated taste cell of claim 213 which does not express a T1R, T2R, or PKD2L1/PKD1L3.

220. The isolated taste cell of claim 213 which expresses a stem cell related gene.

221. The isolated taste cell of claim 213 which expresses a cytokine gene selected from a TNF, MIF, interferon, and an interleukin.

222. The isolated taste cell of claim 213 which expresses a growth factor.

223. The isolated taste cell of claim 213 that expresses gustducin or transducin.

224. The isolated taste cell of claim 213 that does not express gustducin or transducin.

225. A method of using at least one gene contained in Table 1, 2 or 3 or an ortholog or variant thereof encoding a protein at least 90% identical thereto in a cell isolation, purification, enrichment, or marking technique that isolates, purifies, enriches and/or marks at least one desired taste cell subtype or lineage contained in a mixed cell population or cell suspension comprising a desired taste cell type or lineage based on the expression or absence of expression of at least one gene contained in Table 1, 2, or 3, an ortholog thereof, or a gene encoding a protein that is at least 90% identical to said gene or an ortholog thereof.

226. The method of claim 225 wherein the desired taste cell subtype or taste cell lineage is isolated, purified, enriched, or marked by a method that includes the use of a fluorescence activated cell sorter (FACS).

227. The method of claim 225 wherein the desired taste cell subtype or taste cell lineage is isolated, purified, enriched, or marked by a method that includes the use of labeled magnetic beads.

228. The method of claim 225 wherein the mixed cell population or cell suspension is produced by enzymatic digestion and/or tissue disaggregation of tissues containing taste cells.

229. The method of claim 225 wherein the cells are derived from at least one of the tongue, oral cavity, gastrointestinal tract or associated organs, or the urinary tract.

230. The method of claim 225 wherein the desired taste cell subtype or taste cell lineage is isolated, purified, enriched or marked by a method that includes a negative cell selection technique that eliminates at least one non-target taste cell subtype or lineage based on the expression or absence of expression of at least one gene contained in Table 1, 2 or 3.

231. The method of claim 225 wherein the method uses cytotoxic antibodies to specifically kill at least one non-target cell type or lineage.

232. The method of claim 225 wherein the enriched, isolated, purified or marked taste cell subtype or lineage comprises sweet taste cells.

233. The method of claim 225 wherein the enriched, isolated, purified or marked taste cell subtype or lineage comprises umami taste cells.

234. The method of claim 225 wherein the enriched, isolated, purified or marked taste cell subtype or lineage comprises bitter taste cells.

235. The method of claim 225 wherein the enriched, isolated, purified or marked taste cell subtype or lineage comprises sour taste cells.

236. The method of claim 225 wherein the enriched, isolated, purified or marked taste cell subtype or lineage comprises salty taste cells.

237. The method of claim 225 wherein the enriched, isolated, purified or marked taste cell subtype or lineage comprises fat taste cells.

238. The method of claim 225 wherein the enriched, isolated, purified or marked taste cell subtype or lineage comprises taste stem cells.

239. The method of claim 225 wherein the enriched, isolated, purified or marked taste cell subtype or lineage comprises taste cell neurons.

240. The method of claim 225 wherein the enriched, isolated, purified or marked taste cell subtype or lineage comprises taste immune cells.

241. A method of using a cell isolated, purified, enriched or marked according to claim 225 in a method that screens for taste modulatory compounds.

242. A method of using an isolated, enriched, purified or marked taste stem cells produced according to claim 225 in a method that screens for compounds that induce the differentiation of said enriched, isolated, purified or marked taste stem cells into one or more taste cell lineages or subtypes or taste buds.

243. The method of claim 242 wherein said taste cell lineages or subtypes are identified based on the expression or absence of expression of at least one taste specific gene contained in Table 1, 2, or 3.

244. An isolated, purified, enriched or marked taste cell lineage or subtype produced according to claim 225.

245. The isolated, purified, enriched or marked cell lineage or subtype of claim 244 which comprises sweet, umami, bitter, sour, salty, fat, or metallic taste cells or taste cell neurons, immune or stem cells.

246. The isolated, purified, enriched or marked cell lineage or subtype of claim 244 which is human.

247. The isolated, purified, enriched or marked cell lineage or subtype of claim 244 which is derived from human tongue, oral cavity, gastrointestinal tract or associated organs, or the urinary tract or urinary organs.

248. The use of taste specific cells according to claim 244 in an assay that screens for compounds that modulate at least one of sweet, umami, bitter, sour, fat, salty or metallic taste.

249. The use of cells according to claim 244 in a method that screens for compounds that modulate taste cell differentiation or turnover.

250. The use of taste cells according to claim 244 in an assay that screens for compounds that modulate or treat at least one of one of digestive function, taste cell turnover, food sensing, oral immunity, appetite, a gastrointestinal metabolic disorder, nutrient absorption, nutrient excretion, digestive fluid, peptide, enzyme or hormone secretion, taste receptor trafficking, or an autoimmune, neoplastic or inflammatory gastrointestinal disease or disorder.
